(19) United States
(12) Patent Application Publication
(10) Pub. No.: US 2021/0172018 A1
(43) Pub. Date: Jun. 10, 2021

(54) GENETIC VARIANT PANELS AND METHODS OF GENERATION AND USE THEREOF

(71) Applicant: Synthego Corporation, Redwood City, CA (US)

(72) Inventors: Richard Stoner, San Jose, CA (US); Jason Steiner, Redwood City, CA (US); Travis Maures, Pacifica, CA (US); Reed Kelso, San Francisco, CA (US); Aliesha Griffin, San Francisco, CA (US)

(21) Appl. No.: 17/174,902

(22) Filed: Feb. 12, 2021

Related U.S. Application Data
(63) Continuation of application No. PCT/US20/52304, filed on Sep. 23, 2020.
(60) Provisional application No. 62/904,253, filed on Sep. 23, 2019.

(51) Int. Cl. C12Q 1/6883; C12N 15/90

(52) U.S. Cl. CPC C12Q 1/6883; C12Q 2600/156; C12N 15/907

(57) ABSTRACT

Described herein are methods for generating and using variant panels of clonally expanded cells containing a plurality of introduced genetic variants. These clonally expanded cells can be partitioned such that each individual partition contains a single genetic variant, allowing for the assessment of the outcome of each variant without the confounding effect of background genetic variation. Further, panels of such variants can be used to evaluate nucleic acid repair strategies. Genetic variation can be introduced through the use of genome editing tools, such as CRISPR/Cas.

### CROSS-REFERENCE

This application is a continuation of International Application No. PCT/US20/52304, filed Sep. 23, 2020, which claims priority to U.S. provisional patent application No. 62/904,253 filed Sep. 23, 2019, each of which is herein incorporated by reference in its entirety.

### BACKGROUND

Phenotypic traits can show continuous variation due to the influence of multiple loci. In some cases, these loci may be additive in nature, but these loci can interact in ways that are not additive, a phenomenon referred to as epistasis. Even some monogenic traits thought to be under the control of a single locus have been found to be influenced by such epistatic interactions, such as for example through interactions with modifier genes. Therefore, the contribution of a single genetic variant to a particular phenotype can be dependent on the genetic background in which it is found.

Efforts to determine the effect of a large number of genetic variants can rely on methods such as genome wide association studies, which unfortunately can be confounded by the large amount of background genetic variation present in the multitude of individuals required for study. Therefore, a need exists a need for improved, high throughput methods of producing a multitude of genetic variants in a way that limits or substantially eliminates background genetic variation and allows for evaluation of the outcome of each variant. Furthermore, high throughput methods to evaluate different genetic repair strategies can also benefit from the availability of panels of substantially isogenic variants.

### SUMMARY

Described herein, in certain embodiments, are methods for determining one or more outcomes of a plurality of nucleic acid edits, the method comprising: (a) obtaining a plurality of partitions of clonal cells expanded from a plurality of original cells contacted with a plurality of nucleic acid editing units, each nucleic acid editing unit in the plurality of nucleic acid editing units designed to introduce a nucleic acid edit, wherein each partition of clonal cells comprises at least one nucleic acid edit from the plurality of nucleic acid edits; (b) measuring one or more features of cells in each partition of the plurality of clonal cells; and (c) determining one or more outcomes of the nucleic acid edit in each partition of the plurality of clonal cells by comparing the one or more features of cells in each partition of the plurality of clonal cells to one or more features of cells in the plurality of original cells.

In some embodiments, the one or more features of cells in each partition of clonal cells is selected from the group consisting of: a cellular feature, a genetic feature, a gene product feature, a metabolite feature, a lipid feature, and a combination thereof. In some embodiments, one or more features of cells comprise the cellular feature. In some embodiments, the cellular feature is selected from the group consisting of survival, proliferation, viability, cell size, cell shape, cell state, and a combination thereof. In some embodiments, the one or more features of cells comprise the genetic feature. In some embodiments, the genetic feature is selected from the group consisting of a genotype, a haplotype, an epigenetic feature, and a combination thereof. In some embodiments, the epigenetic feature is selected from the group consisting of a presence of an epigenetic modification, a location of the epigenetic modification, an amount of the epigenetic modification, and a combination thereof. In some embodiments, the one or more features of cells comprise the gene product feature. In some embodiments, the gene product feature is selected from the group consisting of a protein expression feature, a protein activity feature, a post-translational modification feature, an RNA expression feature, and a combination thereof. In some embodiments, the protein expression feature is selected from the group consisting of an expression level of a protein, a ratio of expression levels of a plurality of proteins, or a presence or absence of the expression of a protein. In some embodiments, the protein activity feature is a measure of the enzymatic activity of a protein or the binding activity of the protein. In some embodiments, the post-translational modification feature is a presence or absence of a post-translational modification on a protein, a location of the post-translational modification on the protein, or an amount of the post-translational modification on the protein. In some embodiments, the post-translation modification is selected from the group consisting of a phosphorylation, acetylation, glycosylation, amidation, hydroxylation, methylation, ubiquitylation, sulfation, and a combination thereof. In some embodiments, the RNA expression feature is selected from the group consisting of an expression level of an RNA molecule, a ratio of expression levels of a plurality of RNA molecules, or a presence or absence of the expression of an RNA molecule. In some embodiments, the one or more features of the cells comprise the metabolite feature. In some embodiments, the metabolite feature is an amount of one or more metabolites in the cells, a ratio of at least two metabolites in the cells, or a presence or absence of one or more metabolites in the cells. In some embodiments, the one or more features of the cells comprise the lipid feature. In some embodiments, the lipid feature is an amount of one or more lipids in the cells, a ratio of at least two lipids in the cells, or a presence or absence of one or more lipids in the cells.

In some embodiments, each outcome in the one or more outcomes are selected from the group consisting of: a difference in a gene function or no difference in the gene function. In some embodiments, the difference in gene function is an elimination of gene function. In some embodiments, the difference in gene function is a reduction of gene function. In some embodiments, the difference in gene function is an increase in gene function. In some embodiments, the difference in gene function is a restoration of gene function. In some embodiments, the gene function is an activity of a product of a gene.

In some embodiments, the cells in each partition of clonal cells is clonally expanded from a single cell from the plurality of original cells contacted with a plurality of nucleic acid editing units. In some embodiments, cells in all partitions of clonal cells of the plurality of partitions of clonal cells are isogenic outside of the genomic region of interest. In some embodiments, the cells in all partitions of clonal cells of the plurality of partitions of clonal cells are at least 99%, 99.9%, or 99.99% identical outside of the genomic region of interest. In some embodiments, each partition of clonal cells comprises a single nucleic acid edit from the plurality of nucleic acid edits.

In some embodiments, the method further comprises measuring the one or more features of cells in the plurality of original cells. In some embodiments, the genomic region of interest is a gene. In some embodiments, the gene is a human gene. In some embodiments, the human gene is a gene associated with a disease or a modifier of the gene associated with the disease. In some embodiments, the disease is selected from the group consisting of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis, and Von Hippel-Lindau syndrome.

In some embodiments, the plurality of original cells are mammalian cells. In some embodiments, the mammalian cells are selected from the group consisting of: human cells, non-human primate cells, mouse cells, rat cells, rabbit cells, guinea pig cells, hamster cells, cat cells, dog cells, or chicken cells. In some embodiments, the plurality of original cells is from a cell line. In some embodiments, the cell line is selected from the group consisting of: Chinese hamster ovary (CHO) cell line, HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line, and Molt 4 cell line.

Described herein, in certain embodiments, are methods for modifying one or more outcomes of a plurality of first nucleic acid edits in a first genomic region of interest, the method comprising: (a) obtaining a plurality of partitions of clonal cells, wherein each partition of clonal cells comprises a first nucleic acid edit from the plurality of first nucleic acid edits in a first genomic region of interest; and (b) contacting each partition of clonal cells with a second nucleic acid editing unit from a plurality of second nucleic acid editing units, wherein each second nucleic acid editing unit of the plurality of second nucleic acid editing units is designed to introduce a second nucleic acid edit from a plurality of second nucleic acid edits into a second genomic region of interest thereby producing a plurality of partitions of twice edited cells, and wherein an outcome of the first nucleic acid edit is different from an outcome of the second nucleic acid edit. In some embodiments, the first genomic region of interest and the second genomic region of interest are identical.

In some embodiments, the method further comprises measuring one or more features of cells in each partition of twice edited cells. In some embodiments, the one or more features of cells in each partition of twice edited cells are selected from the group consisting of: a cellular feature, a genetic feature, a gene product feature, a metabolite feature, a lipid feature, and a combination thereof.

In some embodiments, one or more features of cells comprise the cellular feature. In some embodiments, the cellular feature is selected from the group consisting of survival, proliferation, viability, cell size, cell shape, cell state, and a combination thereof. In some embodiments, the one or more features of cells comprise the genetic feature. In some embodiments, the genetic feature is selected from the group consisting of a genotype, a haplotype, an epigenetic feature, and a combination thereof. In some embodiments, the epigenetic feature is selected from the group consisting of a presence of an epigenetic modification, a location of the epigenetic modification, an amount of the epigenetic modification, and a combination thereof. In some embodiments, the one or more features of cells comprise the gene product feature. In some embodiments, the gene product feature is selected from the group consisting of a protein expression feature, a protein activity feature, a post-translational modification feature, an RNA expression feature, and a combination thereof. In some embodiments, the protein expression feature is selected from the group consisting of an expression level of a protein, a ratio of expression levels of a plurality of proteins, or a presence or absence of the expression of a protein. In some embodiments, the protein activity feature is a measure of the enzymatic activity of a protein or the binding activity of the protein. In some embodiments, the post-translational modification feature is a presence or absence of a post-translational modification on a protein, a location of the post-translational modification on the protein, or an amount of the post-translational modification on the protein. In some embodiments, the post-translation modification is selected from the group consisting of a phosphorylation, acetylation, glycosylation, amidation, hydroxylation, methylation, ubiquitylation, sulfation, and a combination thereof. In some embodiments, the RNA expression feature is selected from the group consisting of an expression level of an RNA molecule, a ratio of expression levels of a plurality of RNA molecules, or a presence or absence of the expression of an RNA molecule. In some embodiments, the one or more features of the cells comprise the metabolite feature. In some embodiments, the metabolite feature is an amount of one or more metabolites in the cells, a ratio of at least two metabolites in the cells, or a presence or absence of one or more metabolites in the cells. In some embodiments, the one or more features of the cells comprise the lipid feature. In some embodiments, the lipid feature is an amount of one or more lipids in the cells, a ratio of at least two lipids in the cells, or a presence or absence of one or more lipids in the cells.

In some embodiments, the method further comprises measuring one or more features of cells in each partition of clonal cells. In some embodiments, the one or more features of cells in each partition of clonal cells is selected from the group consisting of: a cellular feature, a genetic feature, a gene product feature, a metabolite feature, a lipid feature, and a combination thereof.

In some embodiments, the method further comprises determining an outcome of the second nucleic acid edit in each partition of twice edited cells by comparing the one or more features of cells in each partition of twice edited cells to one or more features of cells in each partition of clonal cells. In some embodiments, each outcome in the one or more outcomes are selected from the group consisting of: a difference in a gene function or no difference in the gene function. In some embodiments, the difference in gene function is an elimination of gene function. In some embodiments, the difference in gene function is a reduction of gene function. In some embodiments, the difference in gene function is an increase in gene function. In some embodiments, the difference in gene function is a restoration of gene function. In some embodiments, the gene function is an activity of a product of a gene.

In some embodiments, the cells in each partition of clonal cells of the plurality of partitions of clonal cells are clonally expanded from a single cell from the plurality of original cells contacted with a plurality of first nucleic acid editing units comprising the plurality of first nucleic acid edits. In some embodiments, the cells in all partitions of the plurality of partitions of clonal cells are isogenic outside of the first genomic region of interest and second genomic region of interest. In some embodiments, the cells in all partitions of clonal cells of the plurality of partitions of clonal cells are at least 99%, 99.9%, or 99.99% identical outside of the genomic region of interest.

In some embodiments, the plurality of original cells is a same cell type. In some embodiments, the cell type is a cell line. In some embodiments, the plurality of original cells is from an individual. In some embodiments, the individual has a disease. In some embodiments, the method further comprises measuring one or more features of cells in the plurality of original cells. In some embodiments, the one or more features of cells in the plurality of original cells are selected from the group consisting of a cellular feature, a genetic feature, a gene product feature, a metabolite feature, a lipid feature, and a combination thereof.

In some embodiments, the method further comprises determining an outcome of the first nucleic acid edit in each partition of clonal cells by comparing the one or more features of cells in each partition of clonal cells to one or more features of cells in the plurality of original cells. In some embodiments, the outcome of the first nucleic acid is selected from the group consisting of: a difference in a gene function or no difference in the gene function. In some embodiments, the difference in gene function is an elimination of gene function. In some embodiments, the difference in gene function is a reduction of gene function. In some embodiments, the difference in gene function is an increase in gene function. In some embodiments, the difference in gene function is a restoration of gene function. In some embodiments, the gene function is an activity of a product of a gene. In some embodiments, the plurality of first nucleic acid edits comprise nucleic acid variants identified in at least one individual having a disease relative to at least one individual not having the disease. In some embodiments, the plurality of first nucleic acid edits comprise nucleic acid variants identified from a database.

In some embodiments, each nucleic acid editing unit in the plurality of nucleic acid editing units comprises an endonuclease and a guide RNA. In some embodiments, the guide RNA is a single guide RNA. In some embodiments, the single guide RNA comprises a guide sequence of about 20 bases and a constant region of from about 22 to about 80 bases in length. In some embodiments, the guide sequence selectively hybridizes to a portion of the second genomic region of interest. In some embodiments, each editing unit in the plurality of editing units further comprises a donor template. In some embodiments, the donor template comprises the nucleic acid edit. In some embodiments, the endonuclease is a Cas protein. In some embodiments, the Cas protein is selected from the group consisting of: Cas9, C2c1, C2c3, and Cpf1. In some embodiments, the endonuclease is a deactivated endonuclease. In some embodiments, the deactivated endonuclease comprises a deactivated endonuclease linked to a deaminase. In some embodiments, the deactivated endonuclease linked to the deaminase is a cytosine base editor or an adenine base editor. In some embodiments, the method further comprises designing the plurality of editing units.

In some embodiments, the second genomic region of interest is a human gene. In some embodiments, the human gene is a gene associated with a disease or a modifier of the gene associated with the disease. In some embodiments, the disease is selected from the group consisting of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis, and Von Hippel-Lindau syndrome.

Described herein, in certain embodiments, are methods for generating a variant panel, the method comprising: (a) obtaining a plurality of partitions of cells from a plurality of original cells contacted with a plurality of nucleic acid editing units, each editing unit designed to introduce at least one nucleic acid edit from a plurality of nucleic acid edits into a genomic region of interest; (b) eliminating substantially all cells except a single cell in each partition of cells of the plurality of partitions; and (c) expanding the single cell in each partition of cells thereby generating a plurality of partitions of clonal cells. In some embodiments, the cells in each partition of cells of the plurality of partitions of cells are isogenic outside of the genomic region of interest. In some embodiments, the cells in all partitions of clonal cells of the plurality of partitions of clonal cells are at least 99%, 99.9%, or 99.99% identical outside of the genomic region of interest. In some embodiments, the cells in each partition of cells of the plurality of partitions of cells are a same cell type. In some embodiments, the cell type is a cell line. In some embodiments, the cells in each partition of cells of the plurality of partitions of cells are from an individual. In some embodiments, the individual has a disease.

In some embodiments, the method further comprises identifying a plurality of nucleic acid variants in the genomic region of interest. In some embodiments, the identifying comprises determining a presence or absence of the plurality of nucleic acid variants in the genomic region of interest from a database. In some embodiments, the identifying comprises determining a presence or absence of the plurality of nucleic acid variants in at least one individual having a disease relative to at least one individual not having the disease. In some embodiments, the plurality of nucleic acid edits comprises the plurality of nucleic acid variants. In some embodiments, the obtaining the plurality of partitions of cells comprises contacting each partition of cells of the plurality of partitions with an editing unit from a plurality of editing units. In some embodiments, at least two, at least three, at least four, or at least five partition of cells of the plurality of partitions are contacted with identical editing units from the plurality of editing units. In some embodiments, each nucleic acid edit in the plurality of nucleic acid edits comprises at least one mutation. In some embodiments, the at least one mutation is a substitution, an insertion, or a deletion. In some embodiments, the plurality of nucleic acid edits comprises at least 4, at least 10, at least 20, at least 30, or at least 50 nucleic acid edits. In some embodiments, the eliminating comprises eliminating all cells except the single cell.

In some embodiments, the method further comprises a first genotyping of cells of each partition of clonal cells of the plurality of partitions, thereby determining a presence or absence of the at least one nucleic acid edit in each partition of clonal cells of the plurality of partitions. In some embodiments, the method further comprises assembling a variant panel comprising a subset of the plurality of partitions of clonal cells comprising a unique genotype as based on the first genotyping. In some embodiments, the method further comprises assembling a variant panel comprising a subset of the plurality of partitions of clonal cells comprising at least one nucleic acid edit based on the determining the presence of the at least one nucleic acid edit.

In some embodiments, the method further comprises repeating steps (a) through (c) when not all of the nucleic acid edits in the plurality of nucleic acid edits are identified in the first genotyping thereby producing a second plurality of partitions of clonal cells. In some embodiments, the method further comprises a second genotyping of cells of each partition of the second plurality of partitions of clonal cells, thereby determining a presence or absence of the at least one nucleic acid edit in each partition of the second plurality of partitions comprising clonal cells. In some embodiments, the method further comprises assembling the variant panel comprising a subset of the plurality of partitions of clonal cells and the second plurality of partitions comprising clonal cells, wherein each partition in the subset of the plurality of partitions of clonal cells comprises a unique genotype as based on the first genotyping and the second genotyping.

In some embodiments, the method further comprises measuring one or more features of cells in each partition of clonal cells. In some embodiments, the method further comprises determining an outcome of the nucleic acid edit in each partition of clonal cells by comparing the one or more features of cells in each partition of clonal cells to one or more features of cells in the plurality of original cells. In some embodiments, the outcome is selected from the group consisting of: a difference in a gene function or no difference in the gene function. In some embodiments, the difference in gene function is an elimination of gene function. In some embodiments, the difference in gene function is a reduction of gene function. In some embodiments, the difference in gene function is an increase in gene function. In some embodiments, the difference in gene function is a restoration of gene function. In some embodiments, the gene function is an activity of a product of a gene.

In some embodiments, the nucleic acid editing unit comprises an endonuclease and a guide RNA. In some embodiments, the guide RNA is a single guide RNA. In some embodiments, the single guide RNA comprises a guide sequence of about 20 bases and further comprises a constant region of from about 22 to about 80 bases in length. In some embodiments, the guide sequence selectively hybridizes to a portion of the genomic region of interest. In some embodiments, the nucleic acid editing unit further comprises a donor template. In some embodiments, the donor template comprises a nucleic acid edit. In some embodiments, each different donor template in a plurality of donor templates comprises a different nucleic acid edit. In some embodiments, the endonuclease is a Cas protein. In some embodiments, the Cas protein is selected from the group consisting of: Cas9, C2c1, C2c3, and Cpf1. In some embodiments, the endonuclease is a deactivated endonuclease. In some embodiments, the deactivated endonuclease comprises a deactivated endonuclease linked to a deaminase. In some embodiments, the deactivated endonuclease linked to the deaminase is a cytosine base editor or an adenine base editor.

In some embodiments, the method further comprises designing the nucleic acid editing unit. In some embodiments, the designing comprises determining a probability distribution of editing outcomes for each potential nucleic acid editing unit of a plurality of potential nucleic acid editing units. In some embodiments, the nucleic acid editing unit is the potential nucleic acid editing unit of the plurality of potential nucleic acid editing units comprising a probability distribution of editing outcomes with a highest probability of introducing the at least one nucleic acid edit from the plurality of nucleic acid edits into the genomic region of interest.

In some embodiments, the genomic region of interest is a human gene. In some embodiments, the human gene is a gene associated with a disease or a modifier of the gene associated with the disease. In some embodiments, the disease is selected from the group consisting of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis, and Von Hippel-Lindau syndrome. In some embodiments, the single cell is a viable cell.

In some embodiments, the eliminating occurs by photoablation of the substantially all cells except the single cell in each partition of the plurality of partitions of cells. In some embodiments, the photoablating occurs at a rate of at least 60 cells per minute. In some embodiments, the photoablating occurs at a rate of at least 90 cells per minute. In some embodiments, the photoablating occurs at a rate of at least 120 cells per minute. In some embodiments, the photoablating comprises using light in the wavelength range of 1440 nm to 1450 nm. In some embodiments, the method further comprises selecting the single cell. In some embodiments, the single cell is based on its position on a surface or in a container. In some embodiments, the selecting the single cell is not based on whether the single cell comprises an exogenous label or an expressed reporter. In some embodiments, the selecting comprises an imaging technique. In some embodiments, the imaging technique comprises bright-field imaging, dark-field imaging, phase contrast imaging, fluorescence imaging, or any combination thereof. In some embodiments, the plurality of partitions of clonal cells are partitioned on a solid support.

Described herein, in certain embodiments, are variant panels comprising a plurality of partitions of clonal cells, each partition of clonal cells comprising a different population of clonal cells designed to have at least one nucleic acid edit from a plurality of at least four nucleic acid edits in a genomic region of interest, and wherein the cells in each partition of cells of the plurality of partitions of cells are isogenic outside of the genomic region of interest. In some embodiments, the cells in all partitions of clonal cells of the plurality of partitions of clonal cells are at least 99%, 99.9%, or 99.99% identical outside of the genomic region of interest. In some embodiments, the cells in each partition of clonal cells of the plurality of partitions are a same cell type. In some embodiments, the cell type is a cell line. In some embodiments, the cells in each partition of the plurality of partitions of clonal cells are from an individual. In some embodiments, the individual has a disease.

In some embodiments, each of the at least four nucleic acid edits are comprised in different partitions of the plurality of partitions. In some embodiments, each of the at least four nucleic acid edits are comprised in at least two, at least three, at least four, or at least five different partitions of the plurality of partitions. In some embodiments, each partition of clonal cells is clonally expanded from a single cell from a plurality of original cells. In some embodiments, the cells in each partition of the plurality of partitions of clonally expanded cells have an outcome selected from the group consisting of: a difference in a gene function or no difference in the gene function, wherein the outcome is determined by comparing one or more features of cells in each partition of clonal cells to one or more features of cells in the plurality of original cells. In some embodiments, the difference in gene function is an elimination of gene function. In some embodiments, the difference in gene function is a reduction of gene function. In some embodiments, the difference in gene function is an increase in gene function. In some embodiments, the difference in gene function is a restoration of gene function. In some embodiments, the gene function is an activity of a product of a gene.

In some embodiments, the one or more features of cells in each partition of clonal cells and one or more features of cells in the plurality of original cells are selected from the group consisting of: a cellular feature, a genetic feature, a gene product feature, a metabolite feature, a lipid feature, and a combination thereof. In some embodiments, the one or more features of cells comprise the cellular feature. In some embodiments, the cellular feature is selected from the group consisting of survival, proliferation, viability, cell size, cell shape, cell state, and a combination thereof. In some embodiments, the one or more features of cells comprise the genetic feature. In some embodiments, the genetic feature is selected from the group consisting of a genotype, a haplotype, an epigenetic feature, and a combination thereof. In some embodiments, the epigenetic feature is selected from the group consisting of a presence of an epigenetic modification, a location of the epigenetic modification, an amount of the epigenetic modification, and a combination thereof. In some embodiments, the one or more features of cells comprise the gene product feature. In some embodiments, the gene product feature is selected from the group consisting of a protein expression feature, a protein activity feature, a post-translational modification feature, an RNA expression feature, and a combination thereof. In some embodiments, the protein expression feature is selected from the group consisting of an expression level of a protein, a ratio of expression levels of a plurality of proteins, or a presence or absence of the expression of a protein. In some embodiments, the protein activity feature is a measure of the enzymatic activity of a protein or the binding activity of the protein. In some embodiments, the post-translational modification feature is a presence or absence of a post-translational modification on a protein, a location of the post-translational modification on the protein, or an amount of the post-translational modification on the protein. In some embodiments, the post-translation modification is selected from the group consisting of a phosphorylation, acetylation, glycosylation, amidation, hydroxylation, methylation, ubiquitylation, sulfation, and a combination thereof. In some embodiments, the RNA expression feature is selected from the group consisting of an expression level of an RNA molecule, a ratio of expression levels of a plurality of RNA molecules, or a presence or absence of the expression of an RNA molecule. In some embodiments, the one or more features of the cells comprise the metabolite feature. In some embodiments, the metabolite feature is an amount of one or more metabolites in the cells, a ratio of at least two metabolites in the cells, or a presence or absence of one or more metabolites in the cells. In some embodiments, the one or more features of the cells comprise the lipid feature. In some embodiments, the lipid feature is an amount of one or more lipids in the cells, a ratio of at least two lipids in the cells, or a presence or absence of one or more lipids in the cells.

In some embodiments, the plurality of partitions of clonally expanded cells are partitioned on a solid support. In some embodiments, the genomic region of interest is a human gene. In some embodiments, the human gene is a gene associated with a disease or a modifier of the gene associated with the disease. In some embodiments, the disease is selected from the group consisting of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis, and Von Hippel-Lindau syndrome. In some embodiments, the plurality of at least four nucleic acid edits comprise at least 10, at least 20, at least 30, or at least 50 nucleic acid edits. In some embodiments, the plurality of at least four nucleic acid edits in the genomic region of interest are identified from a database. In some embodiments, the variant panel is produced by the methods described herein. Described herein, in certain embodiments, are kits comprising the variant panels described herein.

Unlike a pooled approach, the approach of obtaining clonal cells as described herein, which are clonally expanded from a single cell obtained from contacting one or more original cells with the one or more nucleic acid editing units, without employing any kind of selection such as fitness or survival, is bias-free and allows tight control of the genotypes assayed in a panel. Using this approach, Applicant was able to generate a dramatic number of clones with the same genotype, which can then be functionally characterized. Only by capturing all clones in a non-biased fashion, it is possible to get an understanding of all the possible fluctuations between lowest and highest functional activity. This provides a robust way to characterize a functional phenotype, for example, to study the gradations of severity in a disease model. Moreover, in addition to the above, unlike pooled approaches, Applicant's approach can also show the phenotypic differences, if any, between clones with the same genotype. Applicant's unique approach also allows cryopreservation of the clones generated, which can be used to repeat the assays in the future or to do a further in-depth study of the clones at a later time.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1D illustrate the steps of an embodiment of a method described herein for production of a variant panel. FIG. 1A illustrates identification of known variants in a gene. The boxes represent exons, the lines connecting the boxes represent introns, and the arrows represent location of a known variant. FIG. 1B illustrates the location of four variants (V1, V2, V3, and V4) desired to be introduced into a gene, along with corresponding editing units designed to introduce these variants. The nucleic acid editing units comprise four different donor templates (V1 donor, V2 donor, V3 donor, and V4 donor) which contain the nucleic acid edit to be introduced, as well as corresponding single guide RNAs (sgRNA 1, sgRNA 2, sgRNA 3, and sgRNA 4). These editing units are designed to introduce each nucleic acid edit, or variant, into the gene. The single guide RNAs are complexed with a CRISPR protein to produce a ribonucleoprotein (RNP) prior to transfection. FIG. 1C illustrates a multi-well plate containing clonally expanded populations of cells for each of the introduced variants. FIG. 1D illustrates genotyping of the clonal cell population where V1 was the introduced variant, assessment of the function of the clonally expanded V1 variant cells compared to cells with a wild type genotype, and subsequent addition of the V1 variant population to a variant panel.

FIG. 2A-2C illustrate the steps of an embodiment of a method described herein for repair of variants in a variant panel. FIG. 2A illustrates editing units designed to revert, or repair, each of four variants (V1, V2, V3, and V4) to a wild type (WT) genotype. The nucleic acid editing units comprise four different donor templates (V1 donor, V2 donor, V3 donor, and V4 donor) for repair of the variants and four corresponding different single guide RNAs (sgRNA 1, sgRNA 2, sgRNA 3, and sgRNA 4) designed to introduce each repair into a genome. The single guide RNAs are complexed with a CRISPR protein to produce a ribonucleoprotein (RNP) prior to transfection. FIG. 2B illustrates multi-well plates containing twice-edited cell populations. FIG. 2C illustrates assessment of the function of the V1 variant, as determined by amount of a target protein produced, of the twice edited V1 repaired cells compared to wild type (WT) cells and cells with the V1 variant.

FIGS. 3A and 3B illustrate embodiments of methods described herein. FIG. 3A illustrates a method for generating a variant panel described herein. FIG. 3B illustrates a method for modifying an outcome of a plurality of first nucleic acid edits described herein.

FIG. 4 illustrates an embodiment of methods described herein. FIG. 4, in steps 1-12, describes generation of clones and analysis of their functional outcomes.

FIG. 5A-5D illustrate the generation of glucose-6-phosphate dehydrogenase (G6PD) single nucleotide variant (SNV) panel using an embodiment of the methods described herein. FIG. 5A illustrates the 10 SNVs identified from the ClinVar database in the G6PD exon 6. FIG. 5B illustrates all G6PD exon 6 SNV missense mutations and their clinical World Health Organization (WHO) classification, ranging from the most severe (Type I) to normal (Type IV) and variants of unknown clinical significance (VUS). FIG. 5C shows the G6PD clones generated by Synthego's Engineered Cells platform. FIG. 5D shows the homozygous SNV clones and WT control clones generated for functional analysis in the absence of any positive phenotype selection.

FIG. 6A-6C show the phenotype analysis of 14 homozygous single nucleotide G6PD variants generated using an embodiment of the methods described herein. FIG. 6A shows the WHO classification of G6PD deficiency into five different types and their associated clinical presentation. FIG. 6B shows Synthego's Engineered Cells platform generated homozygous SNV clones and wild type (WT) control clone for functional analysis. FIG. 6C illustrates the functional analysis of the 14 G6PD SNV clones. Each box plot represents the percent of wild type (WT) activity for an individual clone.

FIG. 7A-7C illustrate an embodiment of the methods described herein to identify significant phenotype variation between genetically identical clones. FIG. 7A demonstrates the enzymatic activity for homozygous clones generated for the specified G6PD SNV. Each box plot represents the percent of WT activity for an individual clone. The variant score (var score) is the measure of the G6PD activity differences between clones (for example, a var score of 0 means that 0% of the pair-wise comparisons have p-values below 0.01, i.e., 0% of the clone-clone comparison are significantly different from each other, and a var score of 1 means that 100% of the pair-wise comparisons have p-values below 0.01, i.e., 100% of the clone-clone comparison are significantly different from each other). FIG. 7B illustrates the comparison of G6PD activity in all wild type clones generated. FIG. 7C illustrates the comparison of G6PD activity in all G6PD V213L clones. The adjusted p-value of each clone is calculated from comparing the distribution for each clone (for example, an adjusted p-value of 0.01 indicates variable functional activity between clones and an adjusted p-value of closer to 1.00 indicates similar functional activity between clones).

### DETAILED DESCRIPTION

Variant panels and methods of generating such panels, as described herein, can provide the ability to individually assess the outcome of introduced variants in a high throughput manner without the confounding effect of background variation. Furthermore, these variant panels can be used to evaluate a plurality of strategies to repair or further modify these variants.

Described herein, in certain embodiments, are variant panels comprising a plurality of partitions of clonally expanded cells, each partition comprising a different population of clonally expanded cells comprising a nucleic acid edit from a plurality of nucleic acid edits in a genomic region of interest relative to the populations of clonally expanded cells in different partitions in the plurality of partitions, and wherein the cells in each partition of the plurality of partitions are isogenic outside of the genomic region of interest. Further described herein, in certain embodiments, are methods for generating the variant panels described herein. Further described herein, in certain embodiments, are methods for determining an outcome of the plurality of nucleic acid edits. Further described herein, in certain embodiments, are methods for modifying the outcome of the plurality of nucleic acid edits.

Each nucleic acid edit from the plurality of nucleic acid edits can introduce a sequence change, e.g., a mutation, into the genomic region of interest, such as for example, a single nucleotide polymorphism (SNP), a substitution, a deletion, an insertion, duplication, or a copy number variation. A nucleic acid sequence comprising a sequence change relative to a reference nucleic acid sequence or relative to another nucleic acid sequence comprising a different sequence change can also be referred to herein as a variant. The variant can be a naturally occurring variant, such as for example, a mutation associated with a disease.

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a,” “an,” and, “the” include plural referents unless the context clearly dictates otherwise. It is further noted that the claims can be drafted to exclude any optional element. As such, this statement is intended to serve as antecedent basis for use of such exclusive terminology as “solely,” “only,” and the like in connection with the recitation of claim elements or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The term “CRISPR/Cas” or “CRISPR/Cas system” as used herein, can refer to a ribonucleoprotein complex with guide RNA (gRNA) and a CRISPR-associated (Cas) endonuclease. The term “CRISPR” can refer to the Clustered Regularly Interspaced Short Palindromic Repeats and the related system thereof. While CRISPR was discovered as an adaptive defense system that enables bacteria and archaea to detect and silence foreign nucleic acids (e.g., from viruses or plasmids), it can be adapted for use in a variety of cell-types to allow for polynucleotide editing in a sequence-specific manner. In some cases, one or more elements of a CRISPR system can be derived from a type I, type II, type III, or type V CRISPR system. In the CRISPR type II system, the guide RNA can interact with a Cas enzyme and direct the nuclease activity of the Cas enzyme to a target sequence. The target sequence can comprise a “protospacer” and a “protospacer adjacent motif” (PAM), and both domains can be needed for a Cas enzyme mediated activity (e.g., cleavage). The protospacer can be referred to as a cut site (or a genomic target site). The gRNA can pair with (or hybridize) a binding site on the opposite strand of the protospacer to direct the Cas enzyme to the target sequence. The PAM site can generally refer to a short sequence recognized by the Cas enzyme and, in some cases, can be required for the Cas enzyme activity. The sequence and number of nucleotides for the PAM site can differ depending on the type of the Cas enzyme.

The term “Cas,” as used herein, can generally refer to a wild type Cas protein, a fragment thereof, or a mutant or variant thereof.

A Cas protein can comprise a protein of or derived from a CRISPR/Cas type I, type II, or type III system, which can have an RNA-guided polynucleotide-binding or nuclease activity. Examples of suitable Cas proteins include CasX, Cas3, Cas4, Cas5, Cas5e (or CasD), Cash, Cas6e, Cas6f, Cas7, Cas8a1, Cas8a2, Cas8b, Cas8c, Cas9 (also known as Csnl and Csxl2), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, homologues thereof, and modified versions thereof. In some cases, a Cas protein can comprise a protein of or derived from a CRISPR/Cas type V or type VI system, such as Cpf1, C2c1, C2c2, homologues thereof, and modified versions thereof. In some cases, a Cas protein can be a catalytically dead or inactive Cas (dCas).

The term “guide RNA” or “gRNA,” as used herein, can generally refer to an RNA molecule (or a group of RNA molecules collectively) that can bind to a Cas protein and aid in targeting the Cas protein to a specific location within a target polynucleotide (e.g., a DNA). A guide RNA can comprise a CRISPR RNA (crRNA) segment, and optionally a trans-activating crRNA (tracrRNA) segment. The term “crRNA” or “crRNA segment,” as used herein, can refer to an RNA molecule or portion thereof that includes a polynucleotide-targeting guide sequence, a stem sequence, and, optionally, a 5′-overhang sequence. The crRNA can bind to a binding site. The term “tracrRNA” or “tracrRNA segment” can refer to an RNA molecule or portion thereof that includes a protein-binding segment (e.g., the protein-binding segment is capable of interacting with a CRISPR-associated protein, e.g., Cas9). The term “guide RNA” encompasses a single guide RNA (sgRNA), where the crRNA segment and the optional tracrRNA segment are located in the same RNA molecule. The term “guide RNA” also encompasses, collectively, a group of two or more RNA molecules, where the crRNA segment and the tracrRNA segment are located in separate RNA molecules. A guide RNA can comprise nucleotides besides ribonucleotides, e.g., a guide RNA can comprise deoxyribonucleotides.

The term “nucleic acid edit” can refer to a sequence change, such as for example a mutation, substitution, deletion, insertion, duplication, or copy number variation, in a nucleic acid molecule, e.g., a genome, introduced by nucleic acid editing, e.g., genome editing. A non-limiting example of nucleic acid editing, e.g., genome editing, can be CRISPR/Cas genome editing or base editing. The plurality of nucleic acid edits can comprise nucleic acid edits in a genomic region of interest. The plurality of nucleic acid edits in the genomic region of interest can introduce a plurality of disease associated variants. The plurality of nucleic acid edits can comprise from about 4 to about 1000 nucleic acid edits, from about 4 to about 50 nucleic acid edits, from about 20 to about 100 nucleic acid edits, or from about 50 to about 500 nucleic acid edits. The plurality of nucleic acid edits can comprise at least 4, at least 10, at least 20, at least 30, at least 50, at least 100, at least 500, or at least 1000 nucleic acid edits. The plurality of nucleic acid edits can comprise no more than 20, no more than 30, no more than 50, no more than 100, no more than 500, or no more than 1000 nucleic acid edits. In some embodiments, at least one nucleic acid edit in the plurality of nucleic acid edits is a non-naturally occurring variant. In some embodiments, at least one nucleic acid edit in the plurality of nucleic acid edits is a naturally occurring variant.

The term “variant” can refer to a sequence change, such as for example a mutation, substitution, deletion, insertion, duplication, or copy number variation, in a nucleic acid sequence, e.g., genome, relative to a reference nucleic acid sequence, e.g., a reference genome. The variant can be introduced into the genome by genome editing or can be introduced by a process not involving genome editing. A disease associated variant, or a variant in a genome of a cell or individual having a disease, can be identified as a disease associated variant by a comparing the sequence of a cell or individual having the disease to a sequence of a genome of a cell or individual not having the disease. A variant in a genome of a cell or individual having a disease can be identified using at least one database. The at least one database can include gene and/or genome databases comprising sequencing data from DNA and/or RNA. Examples of databases include GENCODE, NCBI, Ensembl, APPRIS, Human Genetic Variation (HGV) database, Catalog of Somatic Mutations in Cancer (COSMIC), HuVarBase, and DisGenNET. The disease associated variant can be a variant in a known disease causing gene or a modifier gene thereof. The plurality of nucleic acid editing units can be designed to generate a plurality of variants from a plurality of original cells. The plurality of nucleic acid editing units can be designed to generate a plurality of disease associated variants from a plurality of original cells. A variant can be a pathogenic variant, a likely pathogenic variant, a variant of uncertain significance, a likely benign variant, or a benign variant.

One or more cells described herein can be subjected to laser photoablation, photoablation, or ablation to disrupt or destroy the one or more cells. The ablation, photoablation, or laser photoablation can comprise exposing light, e.g., intense light, at various wavelengths (ranging from ultraviolet (UV) wavelengths to infrared (IR) wavelengths) in either a pulsed or continuous wave mode to disrupt or destroy the one or more cells.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Variant Panel

Disclosed herein, in certain embodiments, are variant panels comprising: a plurality of partitions of clonally expanded cells, each partition of clonally expanded cells comprising a different population of clonally expanded cells designed to have a nucleic acid edit or combination of nucleic acid edits from a plurality of nucleic acid edits in a genomic region of interest. The plurality of partitions of clonally expanded cells can be clonally expanded from a single cell obtained from a plurality of original cells or a partition of cells from a plurality of original cells contacted with a plurality of nucleic acid editing units, each nucleic acid editing unit designed to introduce a nucleic acid edit from the plurality of nucleic acid edits. The plurality of original cells can be a plurality of wildtype cells.

The population of clonally expanded cells in each partition can be isogenic outside of the genomic region of interest relative to populations of clonally expanded cells in different partitions. In some embodiments, all partitions of populations of clonally expanded cells in the plurality of partitions are isogenic outside of the genomic region of interest. In some cases, isogenic cells are cells that originated or differentiated from the same individual cell or same line of cells. In some cases, the genomes of isogenic cells can be substantially identical except for variation, such as substitutions, insertions, deletions, duplications, or copy number variations that occur as a result of lack of repair of one or more errors from normal cell division. In some cases, isogenic cells comprise essentially identical genomic DNA, for example the genomic DNA or two isogenic cells can be at least about 95%, at least about 96%, at least about 97%, at least about 98%, at least about 99%, at least about 99.5% identical, at least about 99.6% identical, at least about 99.7% identical, at least about 99.8% identical, at least about 99.9% identical, or at least about 99.99% identical.

The plurality of original cells can be any type of cell. The plurality of cells can be eukaryotic cells or prokaryotic cells. The plurality of original cells can be plant cells. The plurality of original cells can be animal cells. The plurality of original cells can be non-mammalian cells. The plurality of original cells can be bacteria. The plurality of original cells can be mammalian cells. The mammalian cells can be human cells, non-human primate cells, mouse cells, rat cells, rabbit cells, guinea pig cells, hamster cells, cat cells, dog cells, cow cells, horse cells, or pig cells. The non-human primate cells can be rhesus macaque cells or chimpanzee cells. The plurality of original cells can be cells from a cell line. The cell line can be a diseased cell line. The cell line can be a human cell line. The cell line can be a CHO cell line (e.g., CHO-K1), HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line, or Molt 4 cell line. The cell line can be a cell line obtained from a repository. Examples of cell line repositories include cell repositories at The Coriell Institute, the American Type Cell Collection, the RIKEN Bioresource Center Cell Bank, Wi-Cell, Boston University, University of Massachusetts International Stem Cell Registry, University of Connecticut Stem Cell Core, Harvard Stem Cell Institute, and the NIMH Stem Cell Center. The plurality of original cells can be primary cells taken directly from living tissue, e.g., by a biopsy. This tissue can be muscle, epithelial, connective, or nervous. The tissue can be from an organ. The organ can be brain, lung, liver, bladder, kidney, heart, stomach, small intestine, large intestine, gallbladder, pancreas, ovary, testes, prostate, eye, ear, or skin. The population of clonally expanded cells in each partition of the plurality of partitions can have originated from a cell or cells from an individual. The individual can have a disease. The cells in the plurality of partitions of clonally expanded cells can have originated from a single cell. The single cell can be from an individual. The individual can be a human. The single cell can be from a cell line. Examples of other cells applicable to the scope of the present disclosure can include stem cells, embryonic stem cells (ESCs) and induced pluripotent stem cells (iPSCs).

In some embodiments, each partition of clonally expanded cells from the plurality of partitions of clonally expanded cells comprises a different variant from a plurality of variants. In some embodiments, each partition of clonally expanded cells from the plurality of partitions of clonally expanded cells comprises a different variant from a plurality of variants. Each variant in the plurality of variants can be produced by a nucleic acid edit from a plurality of nucleic acid edits. In other embodiments, at least two partitions of clonally expanded cells from the plurality of partitions of clonally comprise identical variants. The identical variants can be generated by clonal expansion of different single cells. In some embodiments, at least 2, 3, 4, 5, 6, 7, 8, 9, 10, or more than 10 partitions of clonally expanded cells from the plurality of partitions of clonally expanded cells comprise identical variants generated by clonal expansion of different single cells. In certain embodiments, the partitions of clonally expanded cells may comprise from about 4 to about 1000, from about 4 to about 50, from about 20 to about 100, or from about 50 to about 500 partitions. In some embodiments, the partitions of clonally expanded cells can comprise at least 4, at least 10, at least 20, at least 30, at least 50, at least 100, at least 500, or at least 1000 partitions. In certain embodiments, the partitions of clonally expanded cells can comprise no more than 20, no more than 30, no more than 50, no more than 100, no more than 500, or no more than 1000 partitions. Non-limiting examples of partitions that may be used include containers such as cell culture vessels, e.g., flasks, bottles, bags, multi-well plates etc.

Further disclosed herein, in certain embodiments, are kits comprising a variant panel described herein. The kit can comprise a solid support or a plurality of solid supports. The solid support can be a multi-well plate. The multi-well plate can be a 4-well plate, a 6-well plate, a 12-well plate, a 24-well plate, a 48-well plate, a 96-well plate, or a 384-well plate. In some embodiments, each well in the multi-well plate comprises one partition of clonally expanded cells. In some embodiments, a kit comprises one or more additional containers, each with one or more of various materials (such as reagents, optionally in concentrated form, and/or devices) desirable from a commercial and user standpoint for a use described herein. Examples of such materials include buffers, primers, enzymes, diluents, filters, carrier, package, container, vial and/or tube labels listing contents and/or instructions for use and package inserts with instructions for use. In some cases, a set of instructions is included. In some cases, a label is on or associated with the container. The label can be on a container when letters, numbers or other characters forming the label are attached, molded or etched into the container itself. The label can be associated with a container when it is present within a receptacle or carrier that also holds the container, e.g., as a package insert. The label can be used to indicate that the contents are to be used for a specific application. The label can indicate directions for use of the contents, such as in the methods described herein.

### Creation of a Variant Panel

Described herein, in certain embodiments, are methods for generating a variant panel described herein. A variant panel can comprise a plurality of variants. A variant can be generated by introduction of a nucleic acid edit into the genomic region of interest. Generating a variant panel can comprise: (a) obtaining a plurality of partitions of cells contacted with a plurality of nucleic acid editing units, each editing unit designed to introduce at least one nucleic acid edit from a plurality of nucleic acid edits into a genomic region of interest; (b) eliminating substantially all cells except a single cell in each partition of cells of the plurality of partitions; and (c) expanding the single cell in each partition of cells thereby generating a plurality of partitions of clonally expanded cells. Each partition of clonal cells in the variant panel can be designed to have at least one nucleic acid edit in a genomic region of interest from a plurality of at least two, at least three, at least four, at least five, or at least six nucleic acid edits.

The method can comprise identifying a plurality of variants in the genomic region of interest. The identifying can comprise determining a presence or absence of a plurality of variants in the genomic region of interest. The presence or absence of the plurality of variants can be determined by comparing the sequence of the genomic region of interest to sequence in at least one database. The at least one database can include gene and/or genome databases comprising sequencing data from DNA and/or RNA. Examples of databases include GENCODE, NCBI, Ensembl, APPRIS, Human Genetic Variation (HGV) database, Catalog of Somatic Mutations in Cancer (COSMIC), HuVarBase, and DisGenNET. The plurality of variants can comprise one or more disease-associated variants. The one or more disease-associated variants can be one or more variants identified in an individual having the disease. The one or more disease-associated variants can be one or more variants in an individual with a disease that are not identified in an individual not having the disease. The disease-associated variant can be a pathogenic variant, a likely pathogenic variant, a variant of uncertain significance, a likely benign variant, or a benign variant. The disease-associated variant can be a variant causing the disease or a variant not known to cause the disease. The disease-associated variant can be a variant having an effect on the function of a gene or a variant not known to have an effect on the function of the gene. In some embodiments, each variant in the plurality of variants is a mutation, such as for example, a substitution, an insertion, a deletion, a duplication, or a copy number variation. The plurality of variants can comprise from about 4 to about 1000 variants, from about 4 to about 50 variants, from about 20 to about 100 variants, or from about 50 to about 500 variants. The plurality of variants can comprise at least 4, at least 10, at least 20, at least 30, at least 50, at least 100, at least 500, or at least 1000 variants. The plurality of variants can comprise no more than 20, no more than 30, no more than 50, no more than 100, no more than 500, or no more than 1000 variants. In some embodiments, each partition of clonally expanded cells in the variant panel comprises a single variant from a plurality of variants. In some embodiments, at least one partition of clonally expanded cells in the variant panel comprises at least two variants from a plurality of variants. In some embodiments, at least one partition of clonally expanded cells in the variant panel can comprise 2, 3, 4, 5, 6, 7, 8, 9, or 10 variants. In certain embodiments, the partitions of clonally expanded cells may comprise from about 4 to about 1000, from about 4 to about 50, from about 20 to about 100, or from about 50 to about 500 partitions. In some embodiments, the partitions of clonally expanded cells can comprise at least 4, at least 10, at least 20, at least 30, at least 50, at least 100, at least 500, or at least 1000 partitions. In certain embodiments, the partitions of clonally expanded cells can comprise no more than 20, no more than 30, no more than 50, no more than 100, no more than 500, or no more than 1000 partitions. Non-limiting examples of partitions that may be used include containers such as cell culture vessels, e.g., flasks, bottles, bags, multi-well plates etc.

The genomic region of interest can be nuclear DNA or mitochondrial DNA. The genomic region of interest can be one or more chromosomes, one or more genes, all exons of a gene, all introns of a gene, all of the exons and introns of a gene, all the exons and all of the introns and all of the regulatory sequences of a gene, one or more exons of a gene, one or more introns of a gene, one or more regulatory sequences of a gene, one or more non-coding regions of a gene, all of the sequence of a gene that is transcribed, or any other region affecting expression of a gene. The genomic region of interest can be 1, 2, 3, 4, 5, 6, 7, 8, 9, or 10 exons in a gene. The genomic region of interest can be 1, 2, 3, 4, 5, 6, 7, 8, 9, or 10 introns in a gene. The genomic region of interest can be the first 1, 2, 3, 4, 5, 6, 7, 8, 9, or 10 exons in a gene. The genomic region of interest can be the first 1, 2, 3, 4, 5, 6, 7, 8, 9, or 10 introns in a gene. The genomic region of interest can be the last 1, 2, 3, 4, 5, 6, 7, 8, 9, or 10 exons in a gene. The genomic region of interest can be the last 1, 2, 3, 4, 5, 6, 7, 8, 9, or 10 introns in a gene. The genomic region of interest can be a second gene that interacts with a first gene (i.e., a modifier). A modifier, also referred to as a modifier gene or a genetic modifier, can alter (e.g. suppress, reduce, or increase) the expression of a first gene or alter the expression of a phenotype associated with the first gene. The genomic region of interest can be an exon of the modifier gene, an intron of the modifier gene, a regulatory sequence of the modifier gene, a non-coding region of the modifier gene, or any other region affecting expression of the modifier gene. The genomic region of interest can encode a microRNA (miRNA) affecting expression of a gene or a modifier of the gene. The regulatory sequence can be a promoter, a 5′ untranslated region (5′ UTR), a 3′ untranslated region (3′ UTR), an enhancer, or a silencer. The gene can be a human gene. The human gene can be associated with a disease. The disease can be a cancer. The disease can be a mitochondrial disease. The disease can be a monogenic disease. Examples of monogenic disease can include the diseases shown in TABLE 1. The gene can be a gene associated with a monogenic disease or a modifier gene thereof. Genes associated with monogenic diseases can include the genes shown in TABLE 1.

TABLE 1
Monogenic diseases and associated gene(s)

| Disease | Gene(s) |
|---|---|
| Achondroplasia | Fibroblast growth |
| | factor receptor 3 |
| | (FGFR3) |
| Adrenoleukodystrophy | ATP-binding |
| | cassette, subfamily |
| | D, member 1 |
| | (ABCD1) |
| Alpha thalassaemia | Hemoglobin subunit |
| | alpha 1 (HBA1); |
| | hemoglobin subunit |
| | alpha 2 (HBA2) |
| Alpha-1-antitrypsin | Alpha-1 antitrypsin |
| deficiency | (AAT) |
| Alport syndrome | Collagen type IV |
| | alpha 5 chain |
| | (COL4A5) |
| Amyotrophic lateral | Chromosome 9 open |
| sclerosis (ALS) | reading frame 72 |
| | (C9orf72); TAR |
| | DNA-binding protein |
| | (TARDBP); FUS |
| | RNA-binding protein |
| | (FUS); Superoxide |
| | dismutase 1 (Sod1) |
| Arginase deficiency | Arginine 1(ARG1) |
| Argininosuccinate lyase | Argininosuccinate |
| deficiency | lyase (ASL) |
| Argininosuccinate | Argininosuccinate |
| synthase 1 deficiency | synthase 1 (ASS1) |
| Becker muscular | Dystrophin (DMD) |
| dystrophy (BMD) | |
| Beta thalassemia | Hemoglobin subunit |
| | beta (HBB) |
| Carbamoyl phosphate | Carbamyl-phosphate |
| synthetase I deficiency | synthase 1(CPS1) |
| Charcot-Marie-Tooth | Peripheral myelin |
| disease | protein-22 (PMP-22) |
| Citrin deficiency | Solute carrier family |
| | 25 member 13 |
| | (SLC25A13) |
| Congenital disorder of | Phosphomannomutase- |
| glycosylation type 1a | 2 (PMM2) |
| (PMM2-CDG) | |
| Crouzon syndrome | Fibroblast growth |
| | factor receptor 2 |
| | (FGFT2) |
| Cystic fibrosis (CF) | Cystic fibrosis |
| | transmembrane |
| | conductance |
| | regulator (CFTR) |
| Dravet syndrome | Sodium voltage- |
| | gated channel alpha |
| | subunit 1(SCN1A) |
| Duchenne muscular | Dystrophin (DMD) |
| dystrophy (DMD) | |
| Dystonia 1, Torsion | Torsin Family 1 |
| | Member A (TOR1A) |
| Emery-Dreifuss | Emerin (EMD); Four |
| muscular dystrophy | and a half LIM |
| | domains 1(FHL1); |
| | lamin A/C (LMNA) |
| Facioscapulohumeral | Structural |
| muscular dystrophy | maintenance of |
| | chromosomes |
| | flexible hinge domain |
| | containing 1 |
| | (SMCHD1) |
| Familial adenomatous | APC, WNT signaling |
| polyposis | pathway regulator |
| | (APC) |
| Familial amyloidotic | Transthyretin (TTR) |
| polyneuropathy | |
| Familial dysautonomia | Inhibitor of kappa |
| | light polypeptide |
| | gene enhancer in B- |
| | cells, kinase |
| | complex-associated |
| | protein (IKBKAP) |
| Fanconi anemia | FA complementation |
| | group A (FANCA); |
| | FA complementation |
| | group C (FANCC); |
| | FA complementation |
| | group G (FANCG) |
| Fragile X syndrome | Fragile X mental |
| | retardation 1 (FMR1) |
| Glucose-6-phosphate | Glucose-6-phosphate |
| dehydrogenase | dehydrogenase |
| deficiency | (G6PD) |
| Glutaric aciduria type 1 | Glutaryl-CoA |
| | dehydrogenase |
| | (GCDH) |
| Hemophilia A | Coagulation factor |
| | VIII (F8) |
| Hemophilia B | Coagulation factor IX |
| | (F9) |
| Hemophagocytic | Hemophagocytic |
| lymphohistiocytosis | lymphohistiocytosis |
| | 1 (HPLH1); Perforin |
| | 1 (PRF1); Unc-13 |
| | homolog D |
| | (UNC13D); Syntaxin |
| | 11 (STX11); Syntaxin |
| | binding protein 2 |
| | (STXBP2) |
| Holt-Oram syndrome | T-box 5 (TBX5) |
| Huntington's disease | Huntingtin (HTT) |
| (HD) | |
| Hyperinsulinemic | ATP-binding |
| hypoglycemia | cassette transporter |
| | sub-family C |
| | member 8 |
| | (ABCC8); |
| | Potassium voltage- |
| | gated channel |
| | subfamily J |
| | member 11 |
| | (KCNJ11); |
| | Glucokinase |
| | (GCK); |
| | Hydroxyacyl-CoA |
| | dehydrogenase |
| | (HADH); Insulin |
| | receptor (INSR); |
| | Glutamate |
| | dehdyrogenase 1 |
| | (GLUD1); Solute |
| | carrier family 16 |
| | member |
| | 1(SLC16A1) |
| Hypokalemic | Calcium channel, |
| periodic paralysis | voltage-dependent, |
| | L type, alpha-1S |
| | subunit |
| | (CACNL1A3) |
| Immunodysregulation | Forkhead box P3 |
| polyendocrinopathy | (FOXP3) |
| enteropathy X- | |
| linked (IPEX) | |
| syndrome | |
| Incontinentia | Inhibitor of nuclear |
| pigmenti | factor kappa B |
| | kinase subunit |
| | gamma (IKBKG) |
| Marfan syndrome | Fibrillin-1 (FBN1) |
| Menkes disease | ATPase copper |
| | transporting alpha |
| | (ATP7A) |
| Metachromatic | Arylsulfatase A |
| leukodystrophy | (ARSA) |
| Mucopolysaccharidosis | Iduronate 2- |
| type II (Hunter | sulfatase (IDS) |
| syndrome) | |
| Multiple endocrine | Menin 1 (MEN1); |
| neoplasia | Ret proto-oncogene |
| | (RET); Cyclin |
| | dependent kinase |
| | inhibitor 1B |
| | (CDKN1B) |
| Multiple hereditary | Exostosin-1 |
| exostosis | (EXT1); Exostosin- |
| | 2 (EXT2) |
| Myotonic | DM1 protein |
| dystrophy | kinase (DMPK); |
| | CCHC-type zinc |
| | finger nucleic acid |
| | binding protein |
| | (CNBP) |
| N-acetylglutamate | N-acetylglutamate |
| synthase deficiency | synthase (NAGS) |
| Neurofibromatosis | Neurofibromin 1 |
| type I | (NF1) |
| Neurofibromatosis | Neurofibromin 2 |
| type II | (NF2) |
| Non-syndromic | Stereocilin (STRC) |
| sensorineural | |
| deafness | |
| Norrie syndrome | Norrin cystine knot |
| | growth factor |
| | (NDP) |
| Ornithine | Ornithine |
| transcarbamylase | carbamoyltransferase |
| deficiency | (OTC) |
| Ornithine | Ornithine |
| translocase | translocase |
| deficiency | (ORNT1) |
| Osteogenesis | Collagen type II |
| imperfecta (brittle | alpha 1 chain |
| bone disease) | (COL1A1); |
| | (COL1A2); |
| | (CRTAP); (P3H1) |
| Paroxysmal | Phosphatidylinosito |
| nocturnal | 1 glycan anchor |
| hemoglobinuria | biosynthesis class |
| | A (PIGA) |
| Phenylketonuria | Phenylalanine |
| (PKU) | hydroxylase (PAH) |
| Polycystic kidney | Polycystin 1 |
| disease | (PKD1); polycystin |
| | 2 (PKD2); |
| | fibrocystin |
| | (PKHD1) |
| Pompe disease | Glucosidase alpha, |
| | acid (GAA) |
| Sickle cell anemia | Hemoglobin |
| (SCA) | subunit beta (HBB) |
| Smith-Lemli-Opitz | 7- |
| syndrome | dehydrocholesterol |
| | reductase (DHCR7) |
| Hereditary spastic | Spastin (SPAST) |
| paraplegia | |
| Spinal and bulbar | Androgen receptor |
| muscular atrophy | (AR) |
| Spinal muscular | Survival of motor |
| atrophy | neuron 1, telomeric |
| | (SMN1); Survival |
| | of motor neuron 2, |
| | centromeric |
| | (SMN2) |
| Spinocerebellar | Ataxin-1 (ATXN1); |
| ataxia | Ataxin-2 (ATXN2); |
| | Ataxin-3 (ATXN3); |
| | Ataxin-7 (ATXN3); |
| | calcium voltage- |
| | gated channel |
| | subunit alpha1A |
| | (CACNA1A); |
| | NOP56 |
| | ribonucleoprotein |
| | (NOP56) |
| Spondylometaphyseal | Presequence |
| dysplasia | translocase |
| | associated motor |
| | 16 (PAM16); |
| | collagen type X |
| | alpha 1 chain |
| | (COL10A1); |
| | phosphate |
| | cytindylyltransferase 1, |
| | choline, alpha |
| | (PCTY1A); |
| | glutathione |
| | peroxidase 4 |
| | (GPX4) |
| Tay-Sachs disease | Hexosaminidase |
| | subunit alpha |
| | (HEXA) |
| Treacher Collins | Treacle ribosome |
| syndrome | biogenesis factor 1 |
| | (TCOF1); RNA |
| | polymerase I and |
| | III subunit C |
| | (POLR1C); RNA |
| | polymerase I and |
| | III subunit D |
| | (POLR1D) |
| Tuberous sclerosis | TSC complex |
| | subunit 1 (TSC1); |
| | TSC complex |
| | subunit 2 (TSC2) |
| Von Hippel-Lindau | Von Hippel-Lindau |
| syndrome | tumor suppressor |
| | (VHL) |

The method can comprise contacting a plurality of original cells with a plurality of nucleic acid editing units. Contacting a plurality of original cells with a plurality of nucleic acid editing units can thereby produce a plurality of once edited cells comprising a first nucleic acid edit. Each editing unit can be designed to introduce at least one nucleic acid edit from a plurality of nucleic acid edits into a genomic region of interest. The cells from the plurality of once edited cells can be partitioned onto or into a solid support, thereby generating a plurality of partitions of cells.

The method can comprise partitioning a plurality of original cells onto or into a solid support, thereby generating a plurality of partitions of cells. The method can further comprise contacting each partition of the plurality of partitions of cells with the plurality of nucleic acid editing units or a subset of the plurality of nucleic acid editing units. The contacting can comprise contacting the plurality of original cells or contacting each partition of cells of the plurality of partitions of cells with all nucleic acid editing units in the plurality of nucleic acid editing units or subset of nucleic acid editing units simultaneously.

The subset of the plurality of nucleic acid editing units can be a plurality of nucleic acid editing units which introduce an identical nucleic acid edit but wherein at least two of the nucleic acid editing units comprise a different endonuclease, a different guide RNA, a different donor template, or a combination thereof. The different guide RNA, different donor template, or combination thereof, can differ by length, sequence, or the combination thereof. Two nucleic acid editing units that comprise at least one different endonuclease, different guide RNA, or different donor template but which introduce an identical nucleic acid edit, can be referred to as degenerate nucleic acid editing units.

Contacting a partition of cells with a plurality of nucleic acid editing units can result in the introduction of at least one nucleic acid edit from a plurality of nucleic acid edits into a genomic region of interest. In some embodiments, the introduction of the at least one nucleic acid edit can occur by homology directed repair (HDR) when a nucleic acid editing unit comprises an endonuclease, a guide RNA, and a donor template. In some embodiments, the introduction of the at least one nucleic acid edit can occur by microhomology mediated end joining (MMEJ) or non-homologous end joining (NHEJ) when the editing unit comprises an endonuclease and a guide RNA but does not comprise a donor template. In some embodiments, the introduction can occur by base editing, when the editing unit comprises an endonuclease linked to a deaminase and a guide RNA. The mechanism by which the plurality of nucleic acid editing units introduces the plurality of nucleic acid edits can comprise at least one, two, three, or all of: HDR, MMEG, NHEJ, and base editing.

In some embodiments, the method comprises designing the nucleic acid editing unit. Each editing unit in the plurality of editing units can comprise an endonuclease and a guide RNA. Each editing unit in the plurality of editing units can further comprise a donor template. In some cases, the nucleic acid editing unit does not comprise a donor template. In some cases, at least one nucleic acid editing unit in a plurality of nucleic acid editing units does not comprise a donor template. In some cases, the donor template is not coupled to a complex of the targeted endonuclease with a guide RNA. In some cases, the donor template is not coupled to the complex of the targeted endonuclease with a guide RNA. The donor template can comprise the nucleic acid edit. The method can comprise designing the gRNA, the donor template, or the combination thereof. The gRNA can be designed to have an azimuth score (on target efficiency value) of at least about 0.2, 0.3, 0.4, 0.5, 0.6, 0.7, 0.8, 0.9, or more. The gRNA can be designed to have an azimuth score (on target efficiency value) of greater than 0.4. In some embodiments, the gRNA is designed not to have off-target hybridization sites with 0 or 1 mismatches.

Designing the nucleic acid editing unit can comprise determining a probability distribution of editing outcomes for each potential editing unit of a plurality of potential editing units. The nucleic acid editing unit can be a potential editing unit from the plurality of potential editing units with the highest probability of introducing the at least one nucleic acid edit from the plurality of nucleic acid edits into the genomic region of interest. Introducing the at least one nucleic acid edit can occur as a result of repair of a cut in the genomic sequence caused by the nucleic acid editing unit. In some embodiments, the repair occurs in the presence of the endonuclease and gRNA of the nucleic acid editing unit. In some embodiments, the repair further occurs in the presence of a donor template of the nucleic acid editing unit. In some cases, the nucleic acid editing unit does not comprise a donor template. The repair can be generated by multiple repair mechanisms, such as for example, microhomology mediated end joining (MMEJ), non-homologous end joining (NHEJ), and homology directed repair (HDR). Generating the probability distribution can comprise identifying a plurality of editing events produced by the potential editing unit. The plurality of editing events can produce a plurality of editing outcomes, such as for example, indel length or genotype. Generating the probability distribution can comprise determining an editing outcome feature list for each editing outcome in the plurality of editing outcomes, wherein the editing outcome feature list comprises a measure for at least one feature. Generating the probability distribution can comprise determining a prevalence of each editing outcome in the plurality of editing outcomes, wherein the prevalence of an editing outcome is determined by: (a) deriving a function that transforms the editing outcome feature list of the editing outcome into the prevalence of the editing outcome and (b) applying the function to the editing outcome feature list of the editing outcome to determine the prevalence of the editing outcome. Generating the probability distribution can comprise combining the prevalence of each editing outcome in the plurality of editing outcomes to generate a probability distribution of editing outcomes resulting from repair of the cut in the genomic region of interest by the potential editing unit. The nucleic acid editing unit can be an editing unit of the plurality of potential editing units comprising a probability distribution of editing outcomes with a highest probability of introducing the at least one nucleic acid edit from the plurality of nucleic acid edits into the genomic region of interest.

The measure can be a quantitative measure. The at least one feature can be a flanking sequence feature, a guide sequence feature, a targeted endonuclease feature, a cell feature, a donor polynucleotide feature or a combination thereof. The flanking sequence feature can be a feature of the sequence flanking the cut in the genomic region of interest. The flanking sequence feature can be a nucleotide identity at each nucleotide position in a sequence flanking the cut, a nucleotide motif at each nucleotide position in the sequence flanking the cut, at least one microhomology characteristic in the sequence flanking the cut, a methylation status of at least one CpG site in the sequence flanking the cut, a methylation characteristic in the sequence flanking the cut, a chromatin state of the sequence flanking the cut, or a combination thereof. The sequence flanking the cut can comprise at least 15, 20, 25, or 30 bp of a sequence of the genome on each side of the cut. The guide sequence can be a sequence of a guide RNA that directs the targeted endonuclease to produce the cut in the genome of the cell. The guide sequence can be the entire polynucleotide sequence of a single guide RNA. The guide sequence feature can be a melting temperature of a guide sequence, a GC content of the guide sequence, a modification of the guide RNA, or a combination thereof. The targeted endonuclease feature can be a free-energy change of formation of a complex of the targeted endonuclease with a guide RNA. The targeted endonuclease feature can be the free-energy change is the free-energy change for a CRISPR/Cas system mediated formation of an R-loop structure. The cell feature can be a type of the cell. The type of the cell can be a cell line or a tumor type of the cell. The donor polynucleotide feature can be a nucleotide identity at each nucleotide position in the donor polynucleotide, a nucleotide motif at each nucleotide position in the donor polynucleotide, at least one microhomology characteristic in the donor polynucleotide, a length of an insertion produced by incorporation of the donor polynucleotide in the genome, a nucleotide identity of each nucleotide position in the insertion, a length of donor arms of the donor polynucleotide, a nucleotide identity of each nucleotide position in the donor arms, a nucleotide motif at each nucleotide position in the donor polynucleotide, a GC content of the donor polynucleotide, a melting temperature of the donor polynucleotide, or a combination thereof.

The method can comprise determining a prevalence, or probability, of each editing outcome in the plurality of editing outcomes. The prevalence can be a predicted prevalence. The prevalence of each editing outcome in the plurality of editing outcomes can be determined by deriving a function that transforms the editing outcome feature list of an editing outcome into a prevalence of the editing outcome. The function can be applied to the editing outcome feature list of an editing outcome to determine the prevalence of the editing outcome. Deriving the function can comprise the use of a machine learning model. The machine learning model can use a training data set to derive the function. The training data set can comprise a plurality of editing outcomes generated in vitro or in vivo in a cell or a plurality of cells by a plurality of endonucleases.

Each different donor template in the plurality of donor templates can be designed to introduce a different nucleic acid edit, or variant, via repair of a cut produced by the endonuclease of the nucleic acid editing unit. In some embodiments, at least two donor templates in the plurality of donor templates are designed to introduce a different nucleic acid edit, or variant, via repair of a cut produced by the endonuclease of the nucleic acid editing unit. The repair can homology directed repair (HDR). Each different donor template in the plurality of donor templates can introduce a different nucleic acid edit when contacted, along with a guide RNA and endonuclease, with a cell. The plurality of donor templates can comprise from 1 to 500 donor templates. The plurality of donor polynucleotides can comprise at least 1, 2, 3, 4, 5, 6, 7, 8, 9, 10, 15, 20, 30, 40, 50, 60, 70, 80, 90, 100, 200, 300, 400, or more than 400 donor templates. The plurality of donor templates can comprise at most 500, 400, 300 200, 100, 90, 80, 70, 60, 50, 40, 30, 20, 15, 10, 9, 8, 7, 6, 5, 4, 3, or less gRNAs.

The endonuclease can be a zinc finger nuclease, a transcription activator-like effector nuclease (TALEN), or a Cas endonuclease in a clustered regularly interspaced short palindromic repeats (CRISPR)/CRISPR-associated (Cas) (“CRISPR/Cas”) system. The endonuclease can be a deactivated endonuclease. The Cas in the CRISPR/Cas system can be a type I, type II, type III, or type V Cas. The type II Cas can be Cas9. The type V Cas can be Cpf1. The Cas in the CRISPR/Cas system can be CasX, Cas3, Cas4, Cas5, Cas5e (or CasD), Cash, Cas6e, Cas6f, Cas7, Cas8a1, Cas8a2, Cas8b, Cas8c, Cas9 (also known as Csnl and Csxl2), Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cpf1, C2c1, or C2c. The Cas protein can be Cas9, C2c1, C2c3, or Cpf1. The endonuclease can be a non-naturally occurring endonuclease.

The endonuclease can be a deactivated endonuclease, wherein the deactivated endonuclease lacks the ability to produce a double stranded break (DSB) in a DNA sequence. The endonuclease can be a deactivated Cas (dCas), for example dCas9 or dCpf1. In some embodiments, the deactivated endonuclease is modified to comprise nickase activity, i.e., the ability to produce a single stranded break in the DNA sequence. The deactivated endonuclease can be a Cas nickase. The deactivated endonuclease can further be connected to a deaminase. The deaminase can be a eukaryotic or a prokaryotic deaminase. The deaminase can be a naturally occurring deaminase sequence or a non-naturally occurring deaminase sequence. The deaminase can be a recombinant deaminase. In some embodiments, the deaminase is a cytidine deaminase. The cytidine deaminase can be APOBEC1 or cytosine deaminase 1 (CDA1). In some embodiments, the deaminase is an adenine deaminase. The adenine deaminase can be a transfer RNA adenosine deaminase (TadA). The deaminase can be connected to the N-terminus or the C-terminus of the deactivated endonuclease, such as for example directly or via a linker. The deactivated endonuclease can further be connected to at least one uracil glycosylase inhibitor (UGI). The at least one UGI can be 1, 2, 3, or more than 3 UGI. The at least one UGI can be a naturally occurring UGI sequence or a non-naturally occurring UGI sequence. The at least one UGI can be connected to the N-terminus or the C-terminus of the deactivated endonuclease or the deaminase, such as for example directly or via a linker.

A deactivated endonuclease linked to a deaminase can be referred to as a base editor. A base editor can convert a purine into a different purine, for example an adenine (A) to a guanine (G) or a G to an A. A base editor can convert a pyrimidine into a different pyrimidine, for example a cytosine (C) into a thymine (T) or a T into a C. A base editor can convert a purine into a pyrimidine, for example, an A into a C or T, or a G into a C or T. A base editor can convert a pyrimidine into a purine, for example, C into an A or G, or a T into an A or G. In some embodiments, the base editor is a cytosine base editor (CBE) (i.e. with the ability to convert cytosine to thymine). In some embodiments, the base editor is an adenine base editor (ABE) (i.e. with the ability to convert adenosine to guanidine). A nucleic acid edit described herein can be a conversion of one base into another by a base editor.

The guide ribonucleic acid (gRNA) can be a single guide RNA (sgRNA). In some cases, the sgRNA can be a single polynucleotide chain. The sgRNA can comprise a hybridizing polynucleotide sequence and a second polynucleotide sequence. The hybridizing polynucleotide sequence can hybridize a portion of the genomic region of interest. The hybridizing polynucleotide sequence of the sgRNA can range from 17 to 23 nucleotides. The hybridizing polynucleotide sequence of the sgRNA can be at least 17, 18, 19, 20, 21, 22, 23, or more nucleotides. The hybridizing polynucleotide sequence of the sgRNA can be at most 23, 22, 21, 20, 19, 18, 17, or less nucleotides. In an example, the hybridizing polynucleotide sequence of the gRNA is 20 nucleotides. The second polynucleotide sequence of the single polynucleotide chain sgRNA can interact (bind) with the Cas enzyme. The second polynucleotide sequence can be about 80 nucleotides. The second polynucleotide sequence can be 80 nucleotides. The second polynucleotide sequence can be at least 80, or more nucleotides. The second polynucleotide sequence can be at most 80, or less nucleotides. Overall, the single polynucleotide chain sgRNA can range from 97 to 103 nucleotides. The single polynucleotide chain sgRNA can be at least 97, 98, 99, 100, 101, 102, 103, or more nucleotides. The single polynucleotide chain sgRNA can be at most 103, 102, 101, 100, 99, 98, 97, or less nucleotides. In an example, the single polynucleotide chain sgRNA can be 100 nucleotides. In some cases, the hybridizing polynucleotide sequence and the second polynucleotide sequence are joined by a linker. In some embodiments, the hybridizing polynucleotide is a crRNA and the second polynucleotide sequence is a tracrRNA.

In some embodiments, at least one nucleotide from at least one guide RNA from the plurality of editing units can be modified. Examples of the modification of the at least one nucleotide can include: (a) end modifications, including 5′ end modifications or 3′ end modifications; (b) nucleobase (or “base”) modifications, including replacement or removal of bases; (c) sugar modifications, including modifications at the 2′, 3′, and/or 4′ positions; and (d) backbone modifications, including modification or replacement of the phosphodiester linkages.

Not wishing to be bound by theory, the modification of the at least one nucleotide can provide, for example: (a) improved target specificity; (b) reduced effective concentration of the CRISPR/Cas complex; (c) improved stability of the gRNA (e.g., resistance to ribonucleases (RNases) and/or deoxyribonucleases (DNases)); and (d) decreased immunogenicity. In an example, the at least one nucleotide from the at least one guide RNA in the initial set of guide RNAs can be a 2′-O-methyl nucleotide. Such modification can increase the stability of the gRNA with respect to attack by RNases and/or DNases.

In some cases, a nucleotide sugar modification incorporated into the guide RNA is selected from the group consisting of 2′-O—C1-4alkyl such as 2′-O-methyl (2′-OMe), 2′-deoxy (2′-H), 2′-O—C1-3alkyl-O—C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-unlocked nucleic acid (“ULNA”) nucleotide, a sugar in L form (“L-sugar”), and 4′-thioribosyl nucleotide. In some cases, an internucleotide linkage modification incorporated into the guide RNA is selected from the group consisting of: phosphorothioate “P(S)” (P(S)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2)nCOO—)), alkylphosphonate (P(C1-3alkyl) such as methylphosphonate —P(CH3), boranophosphonate (P(BH3)), and phosphorodithioate (P(S)2).

In some cases, a nucleobase (“base”) modification incorporated into the guide RNA is selected from the group consisting of: 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynyl cytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”), and 5-methyl-2-pyrimidine.

In some cases, one or more isotopic modifications are introduced on the nucleotide sugar, the nucleobase, the phosphodiester linkage and/or the nucleotide phosphates. Such modifications include nucleotides comprising one or more ¹⁵N, ¹³C, ¹⁴C, Deuterium, ³H, ³²P, ¹²⁵I, ¹³¹I atoms or other atoms or elements used as tracers.

In some cases, an “end” modification incorporated into the guide RNA is selected from the group consisting of: PEG (polyethyleneglycol), hydrocarbon linkers (including: heteroatom (O,S,N)-substituted hydrocarbon spacers; halo-substituted hydrocarbon spacers; keto-, carboxyl-, amido-, thionyl-, carbamoyl-, thionocarbamaoyl-containing hydrocarbon spacers), spermine linkers, dyes including fluorescent dyes (for example fluoresceins, rhodamines, cyanines) attached to linkers such as for example 6-fluorescein-hexyl, quenchers (for example dabcyl, BHQ) and other labels (for example biotin, digoxigenin, acridine, streptavidin, avidin, peptides and/or proteins). In some cases, an “end” modification comprises a conjugation (or ligation) of the guide RNA to another molecule comprising an oligonucleotide (comprising deoxynucleotides and/or ribonucleotides), a peptide, a protein, a sugar, an oligosaccharide, a steroid, a lipid, a folic acid, a vitamin and/or other molecule. In some cases, an “end” modification incorporated into the guide RNA is located internally in the guide RNA sequence via a linker such as, for example, 2-(4-butylamidofluorescein)propane-1,3-diol bis(phosphodiester) linker, which is incorporated as a phosphodiester linkage and can be incorporated anywhere between two nucleotides in the guide RNA.

In some cases, the guide RNA can be a complex (e.g., via hydrogen bonds) of a CRISPR RNA (crRNA) segment and a trans-activating crRNA (tracrRNA) segment. The crRNA can comprise a hybridizing polynucleotide sequence and a tracrRNA-binding polynucleotide sequence. The hybridizing polynucleotide sequence can hybridize the portion of the gene (e.g., the selected exon of the selected transcript of the plurality of transcripts of the gene). The hybridizing polynucleotide sequence of the crRNA can range from 17 to 23 nucleotides. The hybridizing polynucleotide sequence of the crRNA can be at least 17, 18, 19, 20, 21, 22, 23, or more nucleotides. The hybridizing polynucleotide sequence of the crRNA can be at most 23, 22, 21, 20, 19, 18, 17, or less nucleotides. In an example, the hybridizing polynucleotide sequence of the crRNA is 20 nucleotides. The tracrRNA-binding polynucleotide sequence of the crRNA can be 22 nucleotides. The tracrRNA-binding polynucleotide sequence of the crRNA can be at least 22, or more nucleotides. The tracrRNA-binding polynucleotide sequence of the crRNA can be at most 22, or less nucleotides. Overall, the crRNA can range from 39 to 45 nucleotides. The crRNA can be at least 39, 40, 41, 42, 43, 44, 45, or more nucleotides. The crRNA can be at most 45, 44, 43, 42, 41, 40, 39, or less nucleotides. The tracrRNA can be 72 nucleotides. The tracrRNA can be at least 72, or more nucleotides. The tracrRNA can be at most 72, or less nucleotides. In an example, the hybridizing polynucleotide sequence of the crRNA is 20 nucleotides, the crRNA is 43 nucleotides, and the respective tracrRNA is 72 nucleotides. In some embodiments, the guide RNA is complexed with the endonuclease to produce a ribonucleoprotein (RNP), also referred to herein as a CRISPR/Cas complex.

In some embodiments, the methods described herein comprise contacting a cell or cells, for example in the plurality of original cells, with a nucleic acid sequence encoding the gRNA, the endonuclease, the donor template, or a combination thereof, wherein the gRNA, the endonuclease, and optionally the donor template are a nucleic acid editing unit described herein. The nucleic acid sequence can be DNA or RNA. The contacting can result in transfection of the nucleic acid sequence into the cell or cells. The nucleic acid sequence encoding the gRNA, the endonuclease, the donor template, or a combination thereof can be delivered to the cell or cells complexed with a lipid in the form of a lipoplex. The nucleic acid sequence encoding the gRNA, the endonuclease, the donor template, or a combination thereof can be delivered to the cell or cells complexed with a polymer in the form of a polyplex. The nucleic acid sequence encoding the gRNA, the endonuclease, the donor template, or a combination thereof can be delivered to the cell or cells via electroporation, nucleofection, microinjection, or hydrodynamic delivery. The nucleic acid sequence encoding the gRNA, the endonuclease, the donor template, or a combination thereof can be delivered to the cell or cells via at least one vector. The at least one vector can be a viral vector or a non-viral vector. The viral vector can be an adeno-associated viral vector (AAV), an adenoviral vector, or a lentiviral vector. The non-viral vector can be a plasmid. In one example, a first vector can encode the gRNA and the endonuclease and a second vector can encode the donor template. In another example, a first vector can encode the gRNA, a second vector can encode the endonuclease, and a third vector can encode the donor template.

In some embodiments, the methods described herein comprise contacting a cell or cells, for example in the plurality of original cells, with a nucleic acid editing unit comprising a ribonucleoprotein (comprising an endonuclease complexed with a gRNA), and optionally, a donor template. In some cases, the ribonucleoprotein is covalently attached to the donor template. In some cases, the ribonucleoprotein is not covalently attached to the donor template. The endonuclease, gRNA, donor template, or combination thereof can be conjugated to a cell penetrating polypeptide. The endonuclease, gRNA, donor template, or the combination thereof can be conjugated to a nanoparticle. The nanoparticle can be a gold nanoparticle.

The CRISPR/Cas complex can create a break in a nucleic acid sequence at a target site. The break can be a double stranded break. The break can be a single stranded break. Repair of the break can occur by microhomology-mediated end joining (MMEJ) or non-homologous end joining (NHEJ). In the presence of a donor template, repair can occur by homology directed repair (HDR), which can result in incorporation of the donor template into the nucleic acid sequence. The incorporation of the donor template into the genome can occur at the site of the break in the nucleic acid sequence at the target site.

In some embodiments, the method comprises eliminating substantially all cells except a single cell in each partition of the plurality of partitions of cells. The plurality of partitions of cells can be a plurality of partitions of once edited cells or a plurality of partitions of twice edited cells. In some embodiments, the eliminating comprises eliminating all cells except a single cell. The single cell can be a selected cell. The single cell can be a viable cell. Existing methods for generating cell clones focus on isolating a single cell in a culture container (i.e., cell singulation) using any of a variety of techniques and technologies for separating the cell from a mixture of cells and can include passage of cells through microfluidic features that subject cells to mechanical stress which decrease the fitness and viability of the cell. In contrast, the methods disclosed herein differ from the cell singulation approaches in that they can bypass the difficulties of depositing a single cell on a surface in a container, and instead can focus on destroying unwanted cells, e.g., once they have settled on a surface or in a container.

In some embodiments, the method comprises selecting the single cell. The selection can occur prior to the eliminating. The single cell can be selected based on its position on the surface or in the container. In some embodiments, the selecting the single cell is not based on whether the single cell comprises an exogenous label or an expressed reporter. The selecting can be based on a proximity of the single cell to a center of the surface or the container. In some instances, the selection of a single cell (or a subset of cells) to retain (or destroy) is made on the basis of selection criteria that are dependent on traits or properties inherent to the cells themselves. Traits or properties inherent to the cells can include cell phenotype, cell morphology, cell size, development stage, the presence or absence of one or more specified biomarkers, and/or a reporter molecule status (e.g. the presence or absence of a green fluorescent protein (GFP) signal). The selecting can comprise an imaging technique. The imaging technique can comprise bright-field imaging, dark-field imaging, phase contrast imaging, fluorescence imaging, or any combination thereof.

The eliminating can occur by photoablating substantially all cells except the single cell in each partition of the plurality of partitions of cells. In some embodiments, the cells in each partition of the plurality of partitions are on a solid support, such as a surface or in a container. The surface can be a partitioned surface. The surface can comprise a surface on a culture plate. The surface can be a bottom interior surface of the culture well plate. The container can be a culture plate well. The surface or container can be a solid support. In some embodiments, the method comprises ablating more than one non-selected cell on the surface or in the container. In some embodiments, more than one non-selected cells comprise about 10 to about 15 non-selected cells. In some embodiments, the more than one non-selected cells consist of 10 to 15 non-selected cells. In some embodiments, the more than one non-selected cells are photoablated with an efficiency of from 90% to 99.9%. In some embodiments, the more than one non-selected cells are photoablated with an efficiency of at least 90%, 91%, 92%, 93%, 94%, 95%, 96%, 97%, 98%, 99%, 99.5%, or 99.9%.

Any of a variety of lasers may be used for photoablation purposes. Examples include diode (or semiconductor) lasers, solid-state lasers, gas lasers, and excimer lasers. The laser used for photoablation of cells can produce continuous wave light, and an electro-optic modulator or electronic shutter can be used to create pulses of light of arbitrarily long duration (e.g., ranging from tens of picoseconds to seconds). The laser light used for photoablation of cells in the disclosed methods and systems can be pulsed at a pulse repetition frequency ranging from about 1 Hz to about 100 MHz, depending on the type of laser used. The laser light irradiance (i.e., the radiant flux (power) delivered per unit area of surface, as measured, e.g., in units of W/cm²) can range from about 0.1 W/cm² to about 10¹⁰ W/cm², depending on the type of laser used and the size of the focal spot at the sample plane. The photoablation can comprise the use of a single laser. The photoablation can comprise the use of two or more lasers operating in parallel such that two or more cells can be ablated in parallel.

In some embodiments, the photoablating occurs at a rate of at least 60 cells per minute. In some embodiments, the photoablating occurs at a rate of at least 90 cells per minute. In some embodiments, the photoablating occurs at a rate of at least 120 cells per minute. In some embodiments, the photoablating comprises using light in the wavelength range of 1440 nm to 1450 nm.

In some embodiments, the method comprises expanding the single cell in each partition to generate a plurality of clonally expanded cells. Expanding a single cell from a plurality of once edited cells can produce a plurality of clonally expanded cells. Expanding a single cell from a plurality of twice edited cells can produce a plurality of twice clonally expanded cells. Once one or more cells have been selected for retention using any of the approaches described above, the remaining unwanted cells are eliminated, such as by photoablation, and the surface or container, e.g., culture plate or culture container, may be returned to a suitable incubator or cell culture chamber for growing clonal populations of the selected cells. In some cases, the one or more cells selected for retention are not cultured following ablation of unwanted cells. In some cases, the one or more cells selected for retention are transferred to another surface or container, e.g., following photoablation of one or more unwanted cells. In some cases, the one or more cells selected for retention are analyzed following photoablation of one or more unwanted cells, e.g., the one or more cells are subjected to single cell analysis, e.g., analysis of nucleic acids of single cell.

In some embodiments, the method comprises genotyping cells of each partition of the plurality of partitions of clonally expanded cells. The genotyping can determine a presence or absence of the at least one variant in each partition of the plurality of partitions comprising clonally expanded cells. The genotyping can comprise sequencing of the genomic region of interest. The genotyping can comprise whole genome sequencing. The genotyping can comprise Sanger sequencing, next generation sequencing (NGS), or a combination thereof. Genotyping the clonally expanded cell population can allow identification of whether the nucleic acid edit was successfully incorporated into the genome, identification of additional variants in the genome, determination of whether the clonally expanded cell population was the result of expansion of a single cell expansion, or a combination thereof.

In some embodiments, the method comprises assembling a variant panel. The variant panel can comprise a subset of the plurality of partitions of clonally expanded cells. Each partition of clonally expanded cells in the subset can comprise a unique genotype as based on the genotyping. Each partition of clonally expanded cells in the subset can be a variant comprising a nucleic acid edit from the plurality of nucleic acid edits. Each partition of clonally expanded cells in the subset can, contain no additional variants outside of the genomic region of interest. Each partition of clonally expanded cells in the subset can be the result of expansion of a single cell.

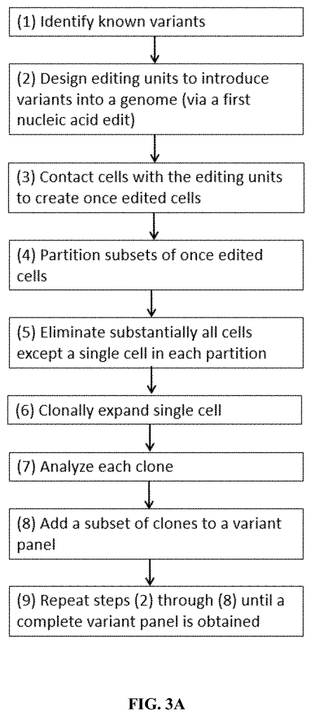

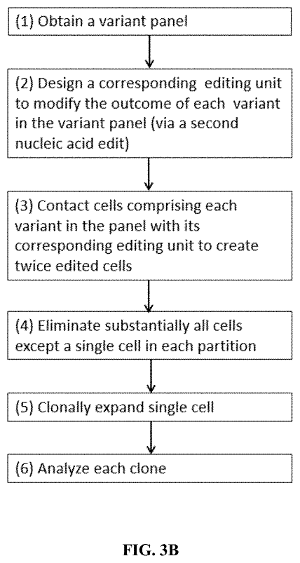

In some embodiments, the method can comprise repeating the steps of: (a) obtaining the plurality of partitions of cells from the plurality of original cells contacted with the plurality of nucleic acid editing units, each editing unit designed to introduce at least one nucleic acid edit from a plurality of nucleic acid edits into a genomic region of interest; (b) eliminating substantially all cells except a single cell in each partition of cells of the plurality of partitions; (c) expanding the single cell in each partition of cells thereby generating a plurality of partitions of clonally expanded cells; and (d) genotyping cells of each partition of the plurality of partitions of clonally expanded cells. These steps can be repeated two, three, four, five, or more than five times. Following repetition of these steps, additional subsets of the additional plurality of partitions of clonally expanded cells can be added to the variant panel. An example of a method for generating a variant panel described herein is illustrated in FIG. 3A.

### Determining Features and Determining Outcomes of an Introduced Nucleic Acid Edit

The methods described herein can comprise determining an outcome of each nucleic acid edit in a plurality of nucleic acid edits in a genomic region of interest. The plurality of nucleic acid edits can be a plurality of first nucleic acid edits, a plurality of second nucleic acid edits, or a combination thereof. The method can comprise obtaining a plurality of partitions of clonally expanded cells from a plurality of original cells contacted with a plurality of nucleic acid editing units, each nucleic acid editing unit in the plurality of nucleic acid editing units designed to introduce a nucleic acid edit, wherein each partition of clonally expanded cells comprises at least one nucleic acid edit from the plurality of nucleic acid edits. The method can comprise obtaining a plurality of partitions twice edited cells generated by contacting each partition of clonally expanded cells from a plurality of partitions of clonally expanded cells with a second nucleic acid editing unit from a plurality of second nucleic acid editing units, each second nucleic acid editing unit designed to introduce a second nucleic acid edit into a genomic region of interest.

Determining Features

In some cases, the methods provided herein can include determining one or more features of one or more cells (e.g., a plurality of cells), one or more tissues, or one or more organisms. The one or more cells, one or more tissues, or one or more organisms can comprise one or more nucleic acid edits introduced by one or more nucleic acid editing units. In some cases, the one or more cells, one or more tissues, or one or more organisms do not comprise one or more nucleic acid edits introduced by one or more nucleic acid editing units. In some cases, the one or more features comprises a quantity. In some cases, the methods provided herein comprise qualifying or quantifying the one or more features. The qualifying can comprise, e.g., determining a presence, an absence, or a category. The quantifying can comprise, e.g., determining an amount, concentration, abundance, rate, or ratio. The one or more features can be one or more cellular features, one or more genetic features, one or more gene product features, one or more metabolite features, or one or more lipid feature. Examples of cellular features, genetic features, gene product features, metabolite features, and lipid features are described herein.

The method can comprise determining one or more features of the cells in each partition of clonally expanded cells, each partition of twice edited cells, the plurality of original cells, or a combination thereof. The one or more features can be determined following incorporation of at least one nucleic acid edit, after clonal expansion, or after a specified period of time has passed from an initial time point, for example 5 minutes, 10 minutes, 15 minutes, 30 minutes, 1 hour, 2 hours, 3 hours, 4 hours, 5 hours, 6 hours, 7 hours, 8 hours, 9 hours, 10 hours, 11 hours, 12 hours, or 24 hours.

A cellular feature can be a quantifiable or qualifiable feature of a cell or a plurality of cells. The cellular feature can be survival, proliferation, viability, cell size, cell shape, cell state, or a combination thereof. In some embodiments, the cellular feature is survival, proliferation, or a combination thereof. Survival of a plurality of cells can comprise a percentage of living cells after the specified period of time. Proliferation of a cell can refer to a change in the number of cells following passage of a specific period of time, i.e., due to cell division. Proliferation of a cell or plurality of cells can be determined by measuring the metabolic activity of the cell, such as by assessing cellular membrane functionality, cytoplasmic enzyme function, or mitochondrial redox potential. Proliferation or survival of a cell or plurality of cells can be determined by counting the number of cells. Prior to counting, the cells can be dyed with a dye, such as trypan blue (TB), nigrosine, eosin, safranin, propidium iodide, 7-aminoactinomycin D, or erythosin B (EB). Cells can be counted manually or with an automated cell counter. Viability of a cell or plurality of cells can be determined by measuring an integrity of a cell membrane, an activity of a cellular enzyme such as an esterase, lactate dehydrogenase, or caspase, or mitochondrial activity. A cell size can be a quantitative or qualitative representation of the size of the cell or the size of a component of the cell. A cell size can include surface area, volume, diameter, radius, circumference, height, width, mass, or a combination thereof. A cell shape can be a feature which can quantitatively or qualitatively describe the shape of a cell. A cell shape can be round, oblong, narrow, flat, tall, jagged, epithelial, branched, square, hexagonal, irregular, or a combination thereof. A cell state can be a quantitative or qualitative description of the current state of the cell. A cell state can be dividing, mitotic, cytokinetic, interphase, gap 1, gap 2, synthesis, senescent, malignant, benign, apoptotic, dead, alive, healthy, or a combination thereof.

A genetic feature can be a quantifiable or qualifiable feature of a genome of a cell or a plurality of cells. The genetic feature can be a genotype, a haplotype, an epigenetic feature, or a combination thereof. A genotype can be a wild type genotype not comprising an introduced nucleic acid edit, or a modified genotype comprising at least one introduced nucleic acid edit (e.g. a first nucleic acid edit or a second nucleic acid edit). The modified genotype can result in a gene knock-out or a gene knock-in. A genotype can be determined by sequencing, PCR, or electrophoresis. The epigenetic feature can comprise a presence of an epigenetic modification, a location of the epigenetic modification, or an amount of the epigenetic modification. The location of the epigenetic modification can be the genomic region of interest. The epigenetic modification can in some instances influence the expression levels of a gene or protein. An epigenetic feature can be a DNA methylation. The DNA methylation can be methylation of a CpG site in the genome. DNA methylation can be determined by bisulfite sequencing.

A gene product feature can be a quantifiable or qualifiable feature of a gene product, such as an mRNA or a protein, in a cell or a plurality of cells. The protein can be an enzyme or a binding protein. The gene product feature can be a protein expression feature, a protein activity feature, a post-translational modification feature, an RNA expression feature, or a combination thereof.

A protein expression feature can include an expression level of a protein, a ratio of expression levels of a plurality of proteins, or a presence or absence of the expression of a protein. The expression level of the protein can be an amount of the protein expressed by a cell or plurality of cells. A protein expression feature can be determined using one or more of flow cytometry, fluorescence activated cell sorting (FACS), magnetic-activated cell sorting (MACS), mass spectroscopy, enzyme-linked immunosorbent assay (ELISA), western blot, sodium dodecyl sulfate-polyacrylamide gel electrophoresis (SDS-PAGE), or other protein expression assay.

A protein activity feature can be a measure of the enzymatic activity or the binding activity of the gene product, such as an enzyme or a binding protein. The measure of the enzymatic activity of an enzyme can comprise determining enzyme activity or determining specific enzyme activity. Determining enzyme activity can comprise determining units of enzyme per ml. Determining specific enzyme activity can comprise determining the amount of substrate the enzyme converts, per mg protein in the enzyme preparation, per unit of time. The protein can be the enzyme.

A post-translational modification feature can be a presence of a post-translational modification, a location of the post-translational modification, or an amount of the post-translational modification. The post-translational modification can be a modification of a protein, and can comprise phosphorylation, acetylation, glycosylation, amidation, hydroxylation, methylation, ubiquitylation, sulfation, or a combination thereof.

An RNA expression feature can include an expression level of an RNA molecule, a ratio of expression levels of a plurality of RNA molecules, or a presence or absence of the expression of an RNA molecule. The expression level of the RNA can be an amount of the RNA expressed by a cell or plurality of cells. RNA protein expression features can be determined using one or more of PCR, electrophoresis, northern blot, in situ hybridization, RNA sequencing, or single cell RNA sequencing.

A metabolite feature can be a quantifiable or qualifiable feature of a metabolite or metabolite profile in a cell or a plurality of cells. The metabolite feature can be an amount of one or more metabolites, a ratio of at least two metabolites, or a presence or absence of one or more lipids. The metabolite can an amino acid, an organic acid, a nucleotide, a fatty acid, an amine, a sugar, a vitamin, a co-factor, a pigment, or an antibiotic.

A lipid feature can be a quantifiable or qualifiable feature of a lipid or lipid profile in a cell or a plurality of cells. The lipid feature can be an amount of one or more lipids, a ratio of at least two lipids, or a presence or absence of one or more lipids. The lipid can be a phospholipid, glycerophospholipid, glycolipid, fatty acid, sphingolipid, sterol lipid (e.g. cholesterol), prenol lipid, or saccharolipid.

Determining Outcomes

The methods provided herein can comprise determining one or more outcomes of the nucleic acid edit in each partition of clonally expanded cells by comparing the one or more features of cells in each partition of clonally expanded cells to one or more features of cells that do not comprise the nucleic acid edit. Cells that do no comprise the nucleic acid edit can be the original cells. The methods can comprise determining one or more outcomes of the second nucleic acid edit in each partition of twice edited cells (or twice clonally expanded cells) by comparing the one or more features of cells in each partition of twice edited cells to one or more features of cells in each partition of clonally expanded cells (or once edited cells). The one or more outcomes of a specific partition of twice edited cells can be compared to the one or more outcomes in a corresponding partition of clonally expanded cells, wherein the corresponding partition of clonally expanded cells is the partition of clonally expanded cells exposed to a specific second editing unit producing the specific partition of twice edited cells. An example of a method for modifying an outcome of a plurality of first nucleic acid edits described herein is illustrated in FIG. 3B.

In some cases, the methods provided herein comprise determining one or more outcomes following introduction of one or more nucleic acid edits into one or more cells, one or more tissues, or one or more organisms. The one or more outcomes can be one or more differences or lack of differences of one or more features of the one or more cells, one or more tissues, or one or more organisms comprising the one or more nucleic acid edits relative to the one or more features of one or more cells, one or more tissues, or one or more organisms that do not comprise the one or more nucleic acid edits. The one or more outcomes can be determined by comparing one or more features of one or more cells, one or more tissues, or one or more organisms comprising one or more nucleic acid edits introduced by one or more nucleic acid editing units relative to the one or more features in one or more cells, one or more tissues, or one or more organisms that do not comprise the one or more nucleic acid edits introduced by one or more nucleic acid editing units. The one or more cells, one or more tissues, or one or more organisms that do not comprise the one or more nucleic acid edits introduced by the one or more nucleic acid editing units can be one or more cells, one or more tissues, or one or more organisms prior to incorporation of the one or more nucleic acid edits. The one or more outcomes can be determined by comparing one or more features of one or more cells comprising a first nucleic acid edit to one or more features of one or more unedited cells. The one or more outcomes can be determined by comparing one or more features of one or more cells comprising a second nucleic acid edit to one or more features of one or more cells comprising a first nucleic acid edit, or one or more unedited cells, or the combination thereof.

The one or more outcomes can be a difference of gene function (e.g., increase, decrease, or restoration to a wildtype gene function) or lack of difference of gene function (e.g., no change). A decrease in gene function can comprise an elimination of gene function. Gene function can be an activity of a product of the gene (i.e. gene product). The activity of the gene product can be an enzymatic activity or a binding activity. The enzymatic activity can be phosphorylation, dephosphorylation, methylation, cleavage, glycosylation, deglycosylation, acetylation, or deacetylation. The binding activity can comprise binding to a protein or a nucleic acid. The protein can be a cell surface receptor, transcription factor, histone, or a ligand of the protein. The nucleic acid can be a single stranded nucleic acid or a double stranded nucleic acid. The nucleic acid can be a DNA or an RNA. The RNA can be an mRNA, tRNA, rRNA, or miRNA. The gene can be a gene comprising the nucleic acid edit or a gene acted upon by a modifier gene comprising the nucleic acid edit. The gene can be an edited gene or an unedited gene. The edited gene can comprise at least 1, 2, 3, 4, 5, 6, 7, 8, 9, 10, or more than 10 nucleic acid edits. The gene can be a gene modified by a modifier gene. The modifier gene can be an edited modifier gene or an unedited modifier gene. The edited modifier gene can comprise at least 1, 2, 3, 4, 5, 6, 7, 8, 9, 10, or more than 10 nucleic acid edits.

The one or more outcomes can be a difference of survival (e.g., increase, decrease, or restoration to a wildtype survival) or lack of difference of survival (no change). A decrease in survival can comprise an elimination of survival. Survival can be a survival of a plurality of cells. Survival can be percentage of survival, an average length of survival, a Kaplan-Meier curve.

The one or more outcomes can be a difference of proliferation (e.g., increase, decrease, or restoration of wildtype proliferation) or lack of difference (no change) of proliferation. A decrease in proliferation can comprise an elimination of proliferation. Proliferation can be a rate of proliferation or a proliferation amount.

In some instances, restoration of wildtype gene function, survival, or proliferation occurs in a one or more cells, one or more tissues, or one or more organisms comprising the one or more second nucleic acid edits, wherein the one or more second nucleic acid edits are designed to correct one or more first nucleic acid edits. The one or more first nucleic acid edits can alter (e.g., increase, decrease, or eliminate) a gene function, survival, or proliferation of one or more wildtype cells, one or more wildtype tissues, or one or more wildtype organisms not comprising the one or more first nucleic acid edits. Restoration of wildtype gene function, survival, or proliferation can comprise a lack of differences of one or more features of the one or more cells, one or more tissues, or one or more organisms comprising the one or more second nucleic acid edits relative to the one or more wildtype cells, one or more wildtype tissues, or one or more wildtype organisms not comprising the one or more first nucleic acid edits. No difference (or a lack of difference) can comprise a difference that is statistically negligible.

In some embodiments, the outcome of the nucleic acid edit, the outcome of the second nucleic acid edit, or a combination thereof can be determined before, during, or after application of a selective pressure to each partition of clonally expanded cells, each partition of twice edited cells (or twice clonally expanded cells), or a combination thereof. The selective pressure can be a biotic selective pressure or an abiotic selective pressure. Application of an abiotic selective pressure can comprise contacting each partition of clonally expanded cells in the plurality of partitions of clonally expanded cells with a therapeutic compound or a compound suspected of having therapeutic activity. Application of an abiotic selective pressure can comprise exposing each partition of clonally expanded cells in the plurality of partitions of clonally expanded cells to a specific environmental condition, such as hypoxia.

Further described herein, in certain embodiments, are methods for screening a test compound on a variant panel described herein. The method can comprise contacting clonally expanded cells in each partition of the plurality of partitions of clonally expanded cells with the test compound. The test compound can be a therapeutic compound or a compound suspected of having therapeutic activity. The method can comprise determining an outcome of the clonally expanded cells in each partition of the plurality of partitions of clonally expanded cells before, after, or before and after the contacting.

### Modification of Outcomes of Introduced Genetic Variation

Described herein, in certain embodiments, are methods for modifying an outcome of a plurality of first nucleic acid edits in a first genomic region of interest. Each partition in a plurality of partitions of clonally expanded cells can comprise a first nucleic acid edit from the plurality of first nucleic acid edits. The plurality of partitions of clonally expanded cells can be a variant panel described herein.

In some embodiments, the method comprises obtaining a plurality of partitions of clonally expanded cells. The plurality of partitions of clonally expanded cells can be obtained following clonal expansion of a cell contacted with a first nucleic acid editing unit from a plurality of first nucleic acid editing units. Each first nucleic acid editing unit in the plurality of first nucleic acid editing units can be designed to introduce a first nucleic acid edit. Each partition of clonally expanded cells can comprise a first nucleic acid edit from the plurality of first nucleic acid edits. The plurality of partitions of clonally expanded cells can be a variant panel described herein. Obtaining the plurality of partitions of clonally expanded cells can comprise generating the variant panel using any of the methods described herein.

In some embodiments, the method comprises contacting each partition of clonally expanded cells with a second nucleic acid editing unit from a plurality of second nucleic acid editing units. Each second nucleic acid editing unit of the plurality of second nucleic acid editing units can be designed to introduce a second nucleic acid edit from a plurality of second nucleic acid edits into a second genomic region of interest thereby producing a plurality of partitions of twice edited cells. The outcome of the first nucleic acid edit can be different from an outcome of the second nucleic acid edit in at least one partition of the plurality of partitions of twice edited cells. The second genomic region of interest can be identical to the first genomic region of interest. The second genomic region of interest can be different from the first genomic region of interest.

A specific second nucleic acid editing unit can be designed for each partition of clonally expanded cells in the plurality of partitions of clonally expanded cells. A specific partition of clonally expanded cells that a specific second nucleic acid editing unit is designed for can be referred to as its corresponding partition of clonally expanded cells, and vice versa. In one example, a specific second nucleic acid editing unit can be designed to repair the nucleic acid edit in its corresponding partition of clonally expanded cells, such that a wild type genotype is produced by successful repair by the specific second editing unit.

The method can comprise determining an outcome of the second nucleic acid edit in each partition of twice edited cells (or twice clonally expanded cells) by comparing the one or more features of cells in each partition of twice edited cells (or twice clonally expanded cells) to one or more features of cells in each partition of clonally expanded cells. The method can comprise determining an outcome of the first nucleic acid edit in each partition of clonally expanded cells by comparing the one or more features of cells in each partition of clonally expanded cells to one or more features of cells in the plurality of original cells. The one or more features of cells in each partition of twice edited cells (or twice clonally expanded cells), each partition of clonally expanded cells, or the plurality of original cells can be determined as previously described herein. The outcome of the second nucleic acid edit or the first nucleic acid edit can be an elimination of gene function, a reduction of gene function, an increase in gene function, or a restoration of gene function.

In some embodiments, an outcome of at least one first nucleic acid edit is different from an outcome of its corresponding second nucleic acid edit. In some example, the outcome of a first nucleic acid edit in a specific partition of clonally expanded cells is an elimination of gene function, a reduction of gene function, an increase in gene function, or a restoration of gene function.

Each second editing unit in the plurality of second editing units can comprise an endonuclease and a guide RNA. Each second editing unit in the plurality of second editing units can further comprise a donor template. Each different donor template in a plurality of donor templates can comprise a different repair of a nucleic acid sequence. Each different donor template in the plurality of donor templates can introduce a different nucleic acid edit when contacted, along with a guide RNA and endonuclease, with a cell comprising a second nucleic acid edit. The plurality of donor templates can comprise from 1 to 500 donor templates. The plurality of donor polynucleotides can comprise at least 1, 2, 3, 4, 5, 6, 7, 8, 9, 10, 15, 20, 30, 40, 50, 60, 70, 80, 90, 100, 200, 300, 400, or more than 400 donor templates. The plurality of donor templates can comprise at most 500, 400, 300 200, 100, 90, 80, 70, 60, 50, 40, 30, 0, 15, 10, 9, 8, 7, 6, 5, 4, 3, or less gRNAs.

In some embodiments, the outcome can be determined after a selective pressure is applied to each partition of clonally expanded cells, each partition of twice edited cells (or twice clonally expanded cells), or a combination thereof. The selective pressure can be a biotic selective pressure or an abiotic selective pressure. Application of an abiotic selective pressure can comprise contacting each partition of clonally expanded cells or each partition of twice edited cells with a therapeutic compound or a compound suspected of having therapeutic activity. Application of an abiotic selective pressure can comprise exposing each partition of clonally expanded cells or each partition of twice edited cells to a specific environmental condition, such as hypoxia. The outcome can be measured before the application of the selective pressure, after the application of the selective pressure, or both before and after the application of the selective pressure.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-258 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method for determining one or more outcomes of one or more nucleic acid edits, the method comprising:
   (a) obtaining one or more partitions of clonal cells, wherein each clonal cell in a partition is clonally expanded from a single cell obtained from contacting one or more original cells with the one or more nucleic acid editing units, and wherein the clonal cells comprise at least one nucleic acid edit in one or more genomic regions of interest; and
   (b) determining one or more outcomes of the one or more nucleic acid edits by comparing one or more features of clonal cells in the partition of clonal cells to one or more features of cells in the one or more original cells.
2. The method of embodiment 1, further comprising comparing one or more features of clonal cells in one partition to one or more features of clonal cells in another partition of a plurality of partitions of clonal cells comprising identical one or more nucleic acid edits in one or more genomic regions of interest.
3. The method of embodiment 1 or 2, wherein the clonal cell is not obtained via a selection.
4. The method of embodiment 3, wherein the selection is based on one or more of: survival, fitness, expression of a protein and expression of an antibiotic.
5. The method of embodiment 4, wherein the protein is a fluorescently labeled protein.
6. The method of any one of embodiments 1-5, wherein the method comprises measuring one or more features of clonal cells in the partition of clonal cells and measuring one or more features of cells in the one or more original cells.
7. The method of any one of embodiments 1-6, wherein the one or more features of clonal cells in the partition of clonal cells and one or more features of cells in the one or more original cells comprise one or more of a cellular feature, a genetic feature, a gene product feature, a metabolite feature and a lipid feature.
8. The method of embodiment 7, wherein the one or more features of cells comprise the cellular feature.
9. The method of embodiment 7 or 8, wherein the cellular feature comprises one or more of: proliferation, viability, cell size, cell shape and cell state.
10. The method of any one of embodiments 7-9, wherein the one or more features of cells comprise the genetic feature.
11. The method of any one of embodiments 7-10, wherein the genetic feature comprises one or more of: a genotype, a haplotype, an epigenetic feature, a presence of a difference in a gene function and an absence of a difference in the gene function.
12. The method of embodiment 11, wherein the difference in gene function is an elimination of gene function.
13. The method of embodiment 11, wherein the difference in gene function is a reduction of gene function.
14. The method of embodiment 11, wherein the difference in gene function is an increase in gene function.
15. The method of embodiment 11, wherein the difference in gene function is a restoration of gene function.
16. The method of any one of embodiments 11-15, wherein the gene function is an activity of a product of a gene.
17. The method of any one of embodiments 11-16, wherein the epigenetic feature comprises one or more of: a presence of an epigenetic modification, an absence of an epigenetic modification, a location of the epigenetic modification and an amount of the epigenetic modification.
18. The method of any one of embodiments 7-17, wherein the one or more features of cells comprise the gene product feature.
19. The method of any one of embodiments 7-18, wherein the gene product feature comprises one or more of: a protein expression feature, a protein activity feature, a post-translational modification feature and an RNA expression feature.
20. The method of embodiment 19, wherein the protein expression feature comprises one or more of: an expression level of a protein, a ratio of expression levels of at least two proteins, a presence of the expression of a protein and an absence of the expression of a protein.
21. The method of embodiment 19, wherein the protein activity feature comprises one or more of: a measure of an enzymatic activity of a protein and a binding activity of the protein.
22. The method of embodiment 19, wherein the post-translational modification feature comprises one or more of: a presence of a post-translational modification on a protein, an absence of a post-translational modification on a protein, a location of the post-translational modification on the protein and an amount of the post-translational modification on the protein.
23. The method of embodiment 22, wherein the post-translation modification comprises one or more of: a phosphorylation, acetylation, glycosylation, amidation, hydroxylation, methylation, ubiquitylation, and sulfation.
24. The method of embodiment 19, wherein the RNA expression feature comprises one or more of: an expression level of an RNA molecule, a ratio of expression levels of at least two RNA molecules, a presence of the expression of an RNA molecule and an absence of the expression of an RNA molecule.
25. The method of any one of embodiments 7-24, wherein the one or more features of the cells comprise the metabolite feature.
26. The method of embodiment 25, wherein the metabolite feature comprises one or more of: an amount of one or more metabolites in the cells, a ratio of at least two metabolites in the cells, a presence of one or more metabolites in the cells and an absence of one or more metabolites in the cells.
27. The method of any one of embodiments 7-26, wherein the one or more features of the cells comprise the lipid feature.
28. The method of embodiment 27, wherein the lipid feature comprises one or more of: an amount of one or more lipids in the cells, a ratio of at least two lipids in the cells, a presence of one or more lipids in the cells and an absence of one or more lipids in the cells.
29. The method of any one of embodiments 1-28, wherein the cells in the one or more partitions of clonal cells are isogenic outside of the one or more genomic regions of interest.
30. The method of any one of embodiments 1-29, wherein the cells in the one or more partitions of clonal cells are at least 99%, 99.9%, or 99.99% identical outside of the one or more genomic regions of interest.
31. The method of any one of embodiments 1-30, wherein each partition of clonal cells comprises a unique genotype.
32. The method of any one of embodiments 1-31, wherein the one or more genomic regions of interest is a gene.
33. The method of embodiment 32, wherein the gene is a human gene.
34. The method of embodiment 33, wherein the human gene is a gene associated with a disease or a modifier of the gene associated with the disease.
35. The method of embodiment 34, wherein the disease comprises one or more of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis and Von Hippel-Lindau syndrome.
36. The method of any one of embodiments 1-35, wherein the partition of clonal cells comprises a single nucleic acid edit from the one or more nucleic acid edits.
37. The method of any one of embodiments 1-36, wherein the one or more nucleic acid edits comprise one or nucleic acid variants.
38. The method of any one of embodiments 1-37, wherein the one or more nucleic acid edits comprise nucleic acid variants identified in at least one individual having a disease relative to at least one individual not having the disease.
39. The method of any one of embodiments 1-38, wherein the one or more nucleic acid edits comprise nucleic acid variants identified from a database.
40. The method of any one of embodiments 1-39, wherein the one or more nucleic acid edits comprise at least one mutation.
41. The method of embodiment 40, wherein the at least one mutation comprises one or more of: a substitution, an insertion, a deletion and a frameshift mutation.
42. The method of any one of embodiments 1-41, wherein the one or more nucleic acid edits comprises at least 4, at least 10, at least 20, at least 30, at least 50, at least 100, at least 250, at least 500 or at least 1000 nucleic acid edits.
43. The method of any one of embodiment 1-42, wherein the one or more original cells are mammalian cells.
44. The method of embodiment 43, wherein the mammalian cells comprise one or more of: human cells, non-human primate cells, mouse cells, rat cells, rabbit cells, guinea pig cells, hamster cells, cat cells, dog cells and chicken cells.
45. The method of embodiment 43 or 44, wherein the mammalian cells are human cells.
46. The method of any one of embodiments 1-42, wherein the one or more original cells is from a cell line.
47. The method of embodiment 46, wherein the cell line comprises one or more of: Chinese hamster ovary (CHO) cell line, HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line and Molt 4 cell line.
48. A method for modifying one or more outcomes of one or more first nucleic acid edits in a first genomic region of interest, the method comprising:
   (a) obtaining one or more partitions of clonal cells, wherein each partition of clonal cells comprises a first nucleic acid edit from the one or more first nucleic acid edits in a first genomic region of interest, and wherein each clonal cell in a partition is clonally expanded from a single cell obtained from contacting one or more original cells with one or more first nucleic acid editing units; and
   (b) contacting each partition of clonal cells with one or more second nucleic acid editing units, wherein each second nucleic acid editing unit is designed to introduce a second nucleic acid edit in a second genomic region of interest thereby producing one or more partitions of twice edited cells, and wherein an outcome of the second nucleic acid edit modifies the outcome of the first nucleic acid edit.
49. The method of embodiment 48, wherein each twice edited cell in a partition is clonally expanded from a single cell obtained from contacting each partition of clonal cells with one or more second nucleic acid editing units.
50. The method of embodiment 48 or 49, wherein the clonal cell is not obtained via a selection.
51. The method of any one of embodiments 48-50, wherein the twice edited cell is not obtained via a selection.
52. The method of embodiment 50 or 51, wherein the selection is based on one or more of: survival, fitness, expression of a protein and expression of an antibiotic.
53. The method of embodiment 52, wherein the protein is a fluorescently labeled protein.
54. The method of any one of embodiments 48-53, wherein the first genomic region of interest and the second genomic region of interest are identical.
55. The method of any one of embodiments 48-53, wherein the first genomic region of interest and the second genomic region of interest are not identical.
56. The method of any one of embodiments 48-55, further comprising measuring one or more features of clonal cells in the one or more partitions of clonal cells.
57. The method of any one of embodiments 48-56, further comprising measuring one or more features of cells in the partition of twice edited cells.
58. The method of any one of embodiments 48-57, further comprising measuring one or more features of the one or more original cells.
59. The method of any one of embodiments 48-58, further comprising determining an outcome of the second nucleic acid edit in the partition of twice edited cells by comparing one or more features of cells in the partition of twice edited cells to one or more features of clonal cells in the partition of clonal cells.
60. The method of any one of embodiments 48-59, further comprising determining an outcome of the second nucleic acid edit in the partition of twice edited cells by comparing one or more features of cells in the partition of twice edited cells to one or more features of cells in the one or more original cells.
61. The method of embodiment 59 or 60, further comprising comparing one or more features of the twice edited cells in one partition to one or more features of the twice edited cells in another partition of a plurality of partitions of twice edited cells comprising an identical second nucleic acid edit in a second genomic region of interest.
62. The method of any one of embodiments 48-61, further comprising determining an outcome of the first nucleic acid edit in the partition of clonal cells by comparing one or more features of clonal cells in the partition of clonal cells to one or more features of cells in the one or more original cells.
63. The method of embodiment 62, further comprising comparing one or more features of clonal cells in one partition to one or more features of clonal cells in another partition of a plurality of partitions of clonal cells comprising an identical first nucleic acid edit in a first genomic region of interest.
64. The method of any one of embodiments 55-63, wherein the one or more features of cells in the partition of twice edited cells, the partition of clonal cells and the one or more original cells comprise one or more of: a cellular feature, a genetic feature, a gene product feature, a metabolite feature and a lipid feature.
65. The method of embodiment 64, wherein the one or more features of cells comprise the cellular feature.
66. The method of embodiment 64 or 65, wherein the cellular feature comprises one or more of: survival, proliferation, viability, cell size, cell shape and cell state.
67. The method of any one of embodiments 64-66, wherein the one or more features of cells comprise the genetic feature.
68. The method of any one of embodiments 64-67, wherein the genetic feature comprises one or more of: a genotype, a haplotype, an epigenetic feature, a presence of a difference in a gene function and an absence of a difference in the gene function.
69. The method of embodiment 68, wherein the difference in gene function is an elimination of gene function.
70. The method of embodiment 68, wherein the difference in gene function is a reduction of gene function.
71. The method of embodiment 68, wherein the difference in gene function is an increase in gene function.
72. The method of embodiment 68, wherein the difference in gene function is a restoration of gene function.
73. The method of any one of embodiments 68-72, wherein the gene function is an activity of a product of a gene.
74. The method of embodiment 68, wherein the epigenetic feature comprises one or more of: a presence of an epigenetic modification, an absence of an epigenetic modification, a location of the epigenetic modification and an amount of the epigenetic modification.
75. The method of any one of embodiments 64-74, wherein the one or more features of cells comprise the gene product feature.
76. The method of any one of embodiments 64-75, wherein the gene product feature comprises one or more of: a protein expression feature, a protein activity feature, a post-translational modification feature and an RNA expression feature.
77. The method of embodiment 76, wherein the protein expression feature comprises one or more of: an expression level of a protein, a ratio of expression levels of at least two proteins, or a presence of the expression of a protein and an absence of the expression of a protein.
78. The method of embodiment 76, wherein the protein activity feature is a measure of an enzymatic activity of a protein or a binding activity of the protein.
79. The method of embodiment 76, wherein the post-translational modification feature is a presence or absence of a post-translational modification on a protein, a location of the post-translational modification on the protein, or an amount of the post-translational modification on the protein.
80. The method of embodiment 79, wherein the post-translation modification comprises one or more of: a phosphorylation, acetylation, glycosylation, amidation, hydroxylation, methylation, ubiquitylation and sulfation.
81. The method of embodiment 76, wherein the RNA expression feature comprises one or more of: an expression level of an RNA molecule, a ratio of expression levels of at least two RNA molecules, a presence the expression of an RNA molecule and an absence of the expression of an RNA molecule.
82. The method of any one of embodiments 64-81, wherein the one or more features of the cells comprise the metabolite feature.
83. The method of embodiment 82, wherein the metabolite feature is an amount of one or more metabolites in the cells, a ratio of at least two metabolites in the cells, or a presence or absence of one or more metabolites in the cells.
84. The method of any one of embodiments 64-83, wherein the one or more features of the cells comprise the lipid feature.
85. The method of embodiment 84, wherein the lipid feature is an amount of one or more lipids in the cells, a ratio of at least two lipids in the cells, or a presence or absence of one or more lipids in the cells.
86. The method of any one of embodiments 48-85, wherein the cells in the one or more partitions of clonal cells are isogenic outside of the first genomic region of interest.
87. The method of any one of embodiments 48-86, wherein the cells in the one or more partitions of clonal cells are at least 99%, 99.9%, or 99.99% identical outside of the first genomic region of interest.
88. The method of any one of embodiments 48-87, wherein the cells in the one or more partitions of twice-edited cells are isogenic outside of the first genomic region of interest and second genomic region of interest.
89. The method of any one of embodiments 48-88, wherein the cells in the one or more partitions of twice-edited cells are at least 99%, 99.9%, or 99.99% identical outside of the first genomic region of interest and second genomic region of interest.
90. The method of any one of embodiments 48-89, wherein each partition of clonal cells comprises a unique genotype.
91. The method of any one of embodiments 48-90, wherein each partition of twice-edited cells comprises a unique genotype.
92. The method of any one of embodiments 48-91, wherein the one or more first nucleic acid edits comprise at least one nucleic acid variant.
93. The method of any one of embodiments 48-91, wherein the one or more first nucleic acid edits comprise nucleic acid variants identified in at least one individual having a disease relative to at least one individual not having the disease.
94. The method of any one of embodiments 48-93, wherein the one or more first nucleic acid edits comprise nucleic acid variants identified from a database.
95. The method of any one of embodiments 48-94, wherein the one or more first nucleic acid edits comprise at least one mutation.
96. The method of embodiment 95, wherein the at least one mutation comprises one or more of: a substitution, an insertion, a deletion and a frameshift mutation.
97. The method of any one of embodiments 48-96, wherein the one or more first nucleic acid edits comprises at least 4, at least 10, at least 20, at least 30, at least 50, at least 100, at least 250, at least 500 or at least 1000 nucleic acid edits.
98. The method of any one of embodiments 48-97, wherein the one or more second nucleic acid edits comprise at least one mutation.
99. The method of embodiment 98, wherein the at least one mutation comprises one or more of: a substitution, an insertion, a deletion and a frameshift mutation.
100. The method of any one of embodiments 48-99, wherein the one or more second nucleic acid edits comprises at least 4, at least 10, at least 20, at least 30, at least 50, at least 100, at least 250, at least 500 or at least 1000 nucleic acid edits.
101. The method of any one of embodiments 48-100, wherein the first genomic region of interest is a gene.
102. The method of any one of embodiments 48-101, wherein the second genomic region of interest is a gene.
103. The method of embodiment 101 or 102, wherein the gene is a human gene.
104. The method of embodiment 103, wherein the human gene is a gene associated with a disease or a modifier of the gene associated with the disease.
105. The method of embodiment 104, wherein the disease comprises one or more of:

achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis and Von Hippel-Lindau syndrome.
106. The method of any one of embodiment 48-105, wherein the one or more original cells are mammalian cells.
107. The method of embodiment 106, wherein the mammalian cells comprise one or more of: human cells, non-human primate cells, mouse cells, rat cells, rabbit cells, guinea pig cells, hamster cells, cat cells, dog cells and chicken cells.
108. The method of embodiment 106 or 107, wherein the mammalian cells are human cells.
109. The method of any one of embodiments 48-105, wherein the one or more original cells is from a cell line.
110. The method of embodiment 109, wherein the cell line comprises one or more of: Chinese hamster ovary (CHO) cell line, HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line and Molt 4 cell line.
111. A method for generating a variant panel, the method comprising:
   (a) contacting one or more original cells with one or more nucleic acid editing units, wherein each editing unit is designed to introduce at least one nucleic acid edit from one or more nucleic acid edits into one or more genomic regions of interest;
   (b) isolating at least one single cell from the one or more original cells contacted with the one or more nucleic acid editing units; and
   (c) expanding each single cell in one or more partitions thereby generating one or more partitions of clonal cells.
112. The method of embodiment 111, wherein the clonal cell is not obtained via a selection.
113. The method of embodiment 112, wherein the selection is based on one or more of: survival, fitness, expression of a protein and expression of an antibiotic.
114. The method of embodiment 113, wherein the protein is a fluorescently labeled protein.
115. The method of any one of embodiments 111-114, wherein the method comprises contacting one or more original cells in one or more partitions with one or more nucleic acid editing units.
116. The method of any one of embodiments 111-115, wherein the cells in the one or more partitions of clonal cells are isogenic outside of the one or more genomic regions of interest.
117. The method of any one of embodiments 111-116, wherein the cells in the one or more partitions of clonal cells are at least 99%, 99.9%, or 99.99% identical outside of the one or more genomic regions of interest.
118. The method of any one of embodiments 111-117, wherein the partition of clonal cells comprises a single nucleic acid edit from the one or more nucleic acid edits.
119. The method of any one of embodiments 111-118, wherein the one or more nucleic acid edits comprise one or nucleic acid variants.
120. The method of any one of embodiments 111-119, wherein the one or more nucleic acid edits comprise nucleic acid variants identified in at least one individual having a disease relative to at least one individual not having the disease.
121. The method of any one of embodiments 111-120, wherein the one or more nucleic acid edits comprise nucleic acid variants identified from a database.
122. The method of any one of embodiments 111-121, wherein each nucleic acid edit in the one or more nucleic acid edits comprises at least one mutation.
123. The method of embodiment 122, wherein the at least one mutation comprises one or more of: a substitution, an insertion, a deletion and a frameshift mutation.
124. The method of any one of embodiments 111-123, wherein the one or more genomic regions of interest is a gene.
125. The method of embodiment 124, wherein the gene is a human gene.
126. The method of embodiment 125, wherein the human gene is a gene associated with a disease or a modifier of the gene associated with the disease.
127. The method of embodiment 126, wherein the disease comprises one or more of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis and Von Hippel-Lindau syndrome.
128. The method of any one of embodiment 111-127, wherein the one or more original cells are mammalian cells.
129. The method of embodiment 128, wherein the mammalian cells comprise one or more of: human cells, non-human primate cells, mouse cells, rat cells, rabbit cells, guinea pig cells, hamster cells, cat cells, dog cells and chicken cells.
130. The method of embodiment 128 or 129, wherein the mammalian cells are human cells.
131. The method of any one of embodiments 111-130, wherein the one or more original cells is from a cell line.
132. The method of embodiment 131, wherein the cell line comprises one or more of: Chinese hamster ovary (CHO) cell line, HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line and Molt 4 cell line.
133. The method of any one of embodiments 111-132, wherein the one or more nucleic acid edits comprises at least 4, at least 10, at least 20, at least 30, at least 50, at least 100, at least 250, at least 500 or at least 1000 nucleic acid edits.
134. The method of any one of embodiments 111-133, further comprising identifying one or more nucleic acid variants in the one or more genomic regions of interest.
135. The method of embodiment 134, wherein the identifying comprises determining a presence or absence of the one or more nucleic acid variants in the one or more genomic regions of interest from a database.
136. The method of embodiment 134 or 135, wherein the identifying comprises determining a presence or absence of the one or more nucleic acid variants in at least one individual having a disease relative to at least one individual not having the disease.
137. The method of any one of embodiments 111-136, further comprising a first genotyping of cells of each partition of clonal cells of the one or more partitions, thereby determining a presence or absence of the at least one nucleic acid edit in each partition of clonal cells of the one or more partitions.
138. The method of embodiment 137, further comprising assembling a variant panel comprising a subset of the one or more partitions of clonal cells, wherein each partition of clonal cells comprises a unique genotype as based on the first genotyping.
139. The method of embodiment 137, further comprising assembling a variant panel comprising a subset of the one or more partitions of clonal cells, wherein each partition of clonal cells comprises at least one nucleic acid edit.
140. The method of any one of embodiments 137-139, further comprising repeating steps (a) through (c), wherein each editing unit of the one or more nucleic acid editing units is designed to introduce at least one nucleic acid edit that was determined to be absent in the first genotyping thereby producing a second one or more partitions of clonal cells.
141. The method of embodiment 140, further comprising a second genotyping of cells of each partition of the second one or more partitions of clonal cells, thereby determining a presence or absence of the at least one nucleic acid edit in each partition of the second one or more partitions comprising clonal cells.
142. The method of embodiment 141, further comprising assembling a variant panel comprising a subset of the one or more partitions of clonal cells and the second one or more partitions comprising clonal cells, wherein each partition of clonal cells comprises a unique genotype as based on the first genotyping and the second genotyping.
143. The method of embodiment 141, further comprising assembling a variant panel comprising a subset of the one or more partitions of clonal cells and the second one or more partitions comprising clonal cells, wherein each partition of clonal cells comprises at least one nucleic acid edit.
144. The method of any one of embodiments 111-143, further comprising measuring one or more features of cells in each partition of clonal cells and measuring one or more features of cells in the one or more original cells.
145. The method of embodiment 143 or 144, wherein the one or more features of clonal cells in the partition of clonal cells and one or more features of cells in the one or more original cells comprise one or more of: a cellular feature, a genetic feature, a gene product feature, a metabolite feature and a lipid feature.
146. The method of embodiment 145, wherein the one or more features of cells comprise the cellular feature.
147. The method of embodiment 145 or 146, wherein the cellular feature comprises one or more of: proliferation, viability, cell size, cell shape and cell state.
148. The method of any one of embodiments 145-147, wherein the one or more features of cells comprise the genetic feature.
149. The method of any one of embodiments 145-148, wherein the genetic feature comprises one or more of: a genotype, a haplotype, an epigenetic feature, a presence of a difference in a gene function and an absence of a difference in the gene function.
150. The method of embodiment 149, wherein the difference in gene function is an elimination of gene function.
151. The method of embodiment 149, wherein the difference in gene function is a reduction of gene function.
152. The method of embodiment 149, wherein the difference in gene function is an increase in gene function.
153. The method of embodiment 149, wherein the difference in gene function is a restoration of gene function.
154. The method of any one of embodiments 149-153, wherein the gene function is an activity of a product of a gene.
155. The method of any one of embodiments 149-154, wherein the epigenetic feature comprises one or more of: a presence of an epigenetic modification, an absence of an epigenetic modification, a location of the epigenetic modification and an amount of the epigenetic modification.
156. The method of any one of embodiments 145-155, wherein the one or more features of cells comprise the gene product feature.
157. The method of any one of embodiments 145-156, wherein the gene product feature comprises one or more of: a protein expression feature, a protein activity feature, a post-translational modification feature and an RNA expression feature.
158. The method of embodiment 157, wherein the protein expression feature comprises one or more of: an expression level of a protein, a ratio of expression levels of at least two proteins, a presence of the expression of a protein and an absence of the expression of a protein.
159. The method of embodiment 157, wherein the protein activity feature comprises one or more of: a measure of an enzymatic activity of a protein and a binding activity of the protein.
160. The method of embodiment 157, wherein the post-translational modification feature comprises one or more of: a presence of a post-translational modification on a protein, an absence of a post-translational modification on a protein, a location of the post-translational modification on the protein and an amount of the post-translational modification on the protein.
161. The method of embodiment 160, wherein the post-translation modification comprises one or more of: a phosphorylation, acetylation, glycosylation, amidation, hydroxylation, methylation, ubiquitylation, and sulfation.
162. The method of embodiment 157, wherein the RNA expression feature comprises one or more of: an expression level of an RNA molecule, a ratio of expression levels of at least two RNA molecules, a presence of the expression of an RNA molecule and an absence of the expression of an RNA molecule.
163. The method of any one of embodiments 145-162, wherein the one or more features of the cells comprise the metabolite feature.
164. The method of embodiment 163, wherein the metabolite feature comprises one or more of: an amount of one or more metabolites in the cells, a ratio of at least two metabolites in the cells, a presence of one or more metabolites in the cells and an absence of one or more metabolites in the cells.
165. The method of any one of embodiments 145-164, wherein the one or more features of the cells comprise the lipid feature.
166. The method of embodiment 165, wherein the lipid feature comprises one or more of: an amount of one or more lipids in the cells, a ratio of at least two lipids in the cells, a presence of one or more lipids in the cells and an absence of one or more lipids in the cells.
167. The method of any one of embodiments 111-166, wherein the one or more genomic regions of interest is a human gene.
168. The method of embodiment 167, wherein the human gene is a gene associated with a disease or a modifier of the gene associated with the disease.
169. The method of embodiment 168, wherein the disease comprises one or more of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis and Von Hippel-Lindau syndrome.
170. The method of any one of embodiments 111-169, wherein the single cell is a viable cell.
171. The method of any one of embodiments 111-170, wherein the single cell is isolated using a single cell printer.
172. The method of any one of embodiments 111-170, wherein the single cell is isolated by photoablation of the substantially all cells except the single cell in each partition of the one or more partitions of cells.
173. The method of embodiment 171, wherein the photoablating occurs at a rate of at least 60 cells per minute.
174. The method of embodiment 171, wherein the photoablating occurs at a rate of at least 90 cells per minute.
175. The method of embodiment 171, wherein the photoablating occurs at a rate of at least 120 cells per minute.
176. The method of embodiment 171, wherein the photoablating comprises using light in the wavelength range of 1440 nm to 1450 nm.
177. The method of any one of embodiments 172-176, further comprising selecting the single cell.
178. The method of embodiment 177, wherein the selecting the single cell is based on its position on a surface or in a container.
179. The method of embodiment 177 or 178, wherein the single cell that is selected does not comprise an exogenous label or an expressed reporter.
180. The method of any one of embodiments 177-179, wherein the single cell is not selected based on binding of an exogenous label or expressing a reporter.
181. The method of any one of embodiments 177-180, wherein the selecting is based on one or more of: a proximity of the cell to a center of a partition, a size of the cell, a morphology of the cell, a phenotype of the cell and a development stage of the cell.
182. The method of any one of embodiments 177-181, wherein the selecting comprises an imaging technique.
183. The method of embodiment 182, wherein the imaging technique comprises one or more of: bright-field imaging, dark-field imaging and phase contrast imaging.
184. The method of embodiment 183, wherein the imaging technique is bright-field imaging.
185. The method of any one of embodiments 1-184, wherein the one or more partitions of clonal cells are partitioned on a solid support.
186. The method of any one of embodiments 1-185, wherein the nucleic acid editing unit comprises an endonuclease and a guide RNA.
187. The method of embodiment 186, wherein the guide RNA comprises a guide sequence that selectively hybridizes to a portion of the one or more genomic regions of interest.
188. The method of embodiment 186 or 187, wherein the guide RNA is a single guide RNA.
189. The method of any one of embodiments 1-188, wherein the nucleic acid editing unit further comprises a donor template.
190. The method of embodiment 189, wherein the donor template comprises a nucleic acid edit.
191. The method of any one of embodiments 186-190, wherein the endonuclease is a CRISPR effector protein.
192. The method of embodiment 191, wherein the CRISPR effector protein is a type II CRISPR effector protein.
193. The method of embodiment 192, wherein the type II CRISPR effector protein is a Cas9 polypeptide.
194. The method of embodiment 191, wherein the CRISPR effector protein is a type V CRISPR effector protein.
195. The method of embodiment 194, wherein the type V CRISPR effector protein is a Cas12a, a Cas12b, a Cas12c, a Cas12d, a Cas12e, a Cas12f, a Cas12g, a Cas12h or a Cas12i polypeptide.
196. The method of embodiment 191, wherein the CRISPR effector protein is a type VI CRISPR effector protein.
197. The method of embodiment 196, wherein the type VI CRISPR effector protein is a Cas13a, a Cas13b, a Cas13c or a Cas13d polypeptide.
198. The method of embodiment 191, wherein the CRISPR effector protein is Cas14a, a Cas14b, or a Cas14c polypeptide.
199. The method of any one of embodiments 186-198, wherein the endonuclease is a deactivated endonuclease.
200. The method of embodiment 199, wherein the deactivated endonuclease comprises a deactivated endonuclease linked to a deaminase.
201. The method of embodiment 200, wherein the deactivated endonuclease linked to the deaminase is a cytosine base editor.
202. The method of embodiment 200, wherein the deactivated endonuclease linked to the deaminase is an adenine base editor.
203. The method of any one of embodiments 1-202, further comprising designing the nucleic acid editing unit.
204. The method of embodiment 203, wherein the designing comprises determining a probability distribution of editing outcomes for each potential nucleic acid editing unit of a plurality of potential nucleic acid editing units.
205. The method of embodiment 204, wherein the nucleic acid editing unit is the potential nucleic acid editing unit of the plurality of potential nucleic acid editing units comprising a probability distribution of editing outcomes with a highest probability of introducing the at least one nucleic acid edit from the plurality of nucleic acid edits into the one or more genomic regions of interest.
206. A variant panel comprising: one or more partitions of clonal cells, wherein each clonal cell in a partition is clonally expanded from a single cell obtained from contacting one or more original cells with the one or more nucleic acid editing units, and wherein the clonal cells comprise at least one nucleic acid edit in one or more genomic regions of interest.
207. The variant panel of embodiment 206, wherein the clonal cell is not obtained via a selection.
208. The variant panel of embodiment 207, wherein the selection is based on one or more of: survival, fitness, expression of a protein and expression of an antibiotic.
209. The variant panel of embodiment 208, wherein the protein is a fluorescently labeled protein.
210. The variant panel of any one of embodiments 206-209, wherein the cells in the one or more partitions of clonal cells are isogenic outside of the one or more genomic regions of interest.
211. The variant panel of any one of embodiments 206-210, wherein the cells in the one or more partitions of clonal cells are at least 99%, 99.9%, or 99.99% identical outside of the one or more genomic regions of interest.
212. The variant panel of any one of embodiments 206-211, wherein the partition of clonal cells comprises a single nucleic acid edit from the one or more nucleic acid edits.
213. The variant panel of any one of embodiments 206-212, wherein the one or more nucleic acid edits comprise one or nucleic acid variants.
214. The variant panel of any one of embodiments 206-213, wherein the one or more nucleic acid edits comprise nucleic acid variants identified in at least one individual having a disease relative to at least one individual not having the disease.
215. The variant panel of any one of embodiments 206-214, wherein the one or more nucleic acid edits comprise nucleic acid variants identified from a database.
216. The variant panel of any one of embodiments 206-215, wherein each nucleic acid edit in the one or more nucleic acid edits comprises at least one mutation.
217. The variant panel of embodiment 216, wherein the at least one mutation comprises one or more of: a substitution, an insertion, a deletion and a frameshift mutation.
218. The variant panel of any one of embodiments 206-217, wherein each partition of clonal cells comprises a unique genotype.
219. The variant panel of any one of embodiments 206-218, wherein the one or more nucleic acid edits comprises at least 4, at least 10, at least 20, at least 30, at least 50, at least 100, at least 250, at least 500 or at least 1000 nucleic acid edits.
220. The variant panel of embodiments 206-219, wherein the one or more genomic regions of interest is a gene.
221. The variant panel of embodiment 220, wherein the gene is a human gene.
222. The variant panel of embodiment 221, wherein the human gene is a gene associated with a disease or a modifier of the gene associated with the disease.
223. The variant panel of embodiment 222, wherein the disease comprises one or more of: achondroplasia, arginase deficiency, argininosuccinate lyase deficiency, argininosuccinate synthase 1 deficiency, adrenoleukodystrophy, alpha thalassaemia, alpha-1-antitrypsin deficiency, Alport syndrome, amyotrophic lateral sclerosis, Becker muscular dystrophy, beta thalassemia, carbamoyl phosphate synthetase I deficiency, Charcot-Marie-Tooth disease, citrin deficiency, congenital disorder of glycosylation type 1a, Crouzon syndrome, cystic fibrosis, Duchenne muscular dystrophy, dystonia 1 Torsion, Emery-Dreifuss muscular dystrophy, facioscapulohumeral muscular dystrophy, familial adenomatous polyposis, familial amyloidotic polyneuropathy, familial dysautonomia, fanconi anaemia, Fragile X syndrome, glucose-6-phosphate dehydrogenase deficiency, glutaric aciduria type 1, hemophilia A, hemophilia B, hemophagocytic lymphohistiocytosis, Holt-Oram syndrome, Huntington's disease, hyperinsulinemic hypoglycemia, hypokalemic periodic paralysis, immunodysregulation polyendocrinopathy enteropathy X-linked (IPEX) syndrome, Incontinentia pigmenti, syndrome, Menkes disease, metachromatic leukodystrophy, mucopolysaccharidosis type II (Hunter syndrome), multiple endocrine neoplasia, multiple hereditary exostosis, myotonic dystrophy, N-acetylglutamate synthase deficiency, neurofibromatosis type I, neurofibromatosis type II, non-syndromic sensorineural deafness, Norrie syndrome, ornithine translocase deficiency, ornithine transcarbamylase deficiency, osteogenesis imperfecta (brittle bone disease), paroxysmal nocturnal hemoglobinuria, polycystic kidney disease, Pompe disease, sickle cell anaemia, Smith-Lemli-Opitz syndrome, hereditary spastic paraplegia, spinal and bulbar muscular atrophy, spinal muscular atrophy, spinocerebellar ataxia, spondylometaphyseal dysplasia, Tay-Sachs disease, Treacher Collins syndrome, tuberous sclerosis and Von Hippel-Lindau syndrome.
224. The variant panel of any one of embodiments 206-223, wherein the one or more clonal cells are mammalian cells.
225. The variant panel of embodiment 224, wherein the mammalian cells comprise one or more of: human cells, non-human primate cells, mouse cells, rat cells, rabbit cells, guinea pig cells, hamster cells, cat cells, dog cells and chicken cells.
226. The variant panel of embodiment 224 or 225, wherein the mammalian cells are human cells.
227. The variant panel of any one of embodiments 206-226, wherein the one or more clonal cells is from a cell line.
228. The variant panel of embodiment 227, wherein the cell line comprises one or more of: Chinese hamster ovary (CHO) cell line, HEK293 cell line, Caco2 cell line, U2-OS cell line, NIH 3T3 cell line, NSO cell line, SP2 cell line, DG44 cell line, K-562 cell line, U-937 cell line, MC5 cell line, IMR90 cell line, Jurkat cell line, HepG2 cell line, HeLa cell line, HT-1080 cell line, HCT-116 cell line, Hu-h7 cell line, Huvec cell line and Molt 4 cell line.
229. The variant panel of any one of embodiments 206-228, wherein the clonal cells in each partition have an outcome determined by comparing one or more features of cells in each partition of clonal cells to one or more features of cells in the one or more of original cells.
230. The variant panel of embodiment 229, wherein the clonal cells in each partition have an outcome further determined by comparing one or more features of clonal cells in one partition to one or more features of clonal cells in another partition of a plurality of partitions of clonal cells comprising identical one or more nucleic acid edits in one or more genomic regions of interest.
231. The variant panel of embodiment 229 or 230, wherein the one or more features of clonal cells in the partition of clonal cells and one or more features of cells in the one or more original cells comprise one or more of: a cellular feature, a genetic feature, a gene product feature, a metabolite feature and a lipid feature.
232. The variant panel of embodiment 231, wherein the one or more features of cells comprise the cellular feature.
233. The variant panel of embodiment 231 or 232, wherein the cellular feature comprises one or more of: proliferation, viability, cell size, cell shape and cell state.
234. The variant panel of any one of embodiments 231-233, wherein the one or more features of cells comprise the genetic feature.
235. The variant panel of any one of embodiments 231-234, wherein the genetic feature comprises one or more of: a genotype, a haplotype, an epigenetic feature, a presence of a difference in a gene function and an absence of a difference in the gene function.
236. The variant panel of embodiment 235, wherein the difference in gene function is an elimination of gene function.
237. The variant panel of embodiment 235, wherein the difference in gene function is a reduction of gene function.
238. The variant panel of embodiment 235, wherein the difference in gene function is an increase in gene function.
239. The variant panel of embodiment 235, wherein the difference in gene function is a restoration of gene function.
240. The variant panel of any one of embodiments 235-239, wherein the gene function is an activity of a product of a gene.
241. The variant panel of embodiment 235, wherein the epigenetic feature comprises one or more of: a presence of an epigenetic modification, an absence of an epigenetic modification, a location of the epigenetic modification and an amount of the epigenetic modification.
242. The variant panel of any one of embodiments 235-241, wherein the one or more features of cells comprise the gene product feature.
243. The variant panel of any one of embodiments 235-242, wherein the gene product feature comprises one or more of: a protein expression feature, a protein activity feature, a post-translational modification feature and an RNA expression feature.
244. The variant panel of embodiment 243, wherein the protein expression feature comprises one or more of: an expression level of a protein, a ratio of expression levels of at least two proteins, a presence of the expression of a protein and an absence of the expression of a protein.
245. The variant panel of embodiment 243, wherein the protein activity feature is a measure of an enzymatic activity of a protein or a binding activity of the protein.
246. The variant panel of embodiment 243, wherein the post-translational modification feature is a presence or absence of a post-translational modification on a protein, a location of the post-translational modification on the protein, or an amount of the post-translational modification on the protein.
247. The variant panel of embodiment 246, wherein the post-translation modification comprises one or more of: a phosphorylation, acetylation, glycosylation, amidation, hydroxylation, methylation, ubiquitylation and sulfation.
248. The variant panel of embodiment 243, wherein the RNA expression feature comprises one or more of: an expression level of an RNA molecule, a ratio of expression levels of at least two RNA molecules, a presence of the expression of an RNA molecule and an absence of the expression of an RNA molecule.
249. The variant panel of any one of embodiments 235-248, wherein the one or more features of the cells comprise the metabolite feature.
250. The variant panel of embodiment 249, wherein the metabolite feature is an amount of one or more metabolites in the cells, a ratio of at least two metabolites in the cells, or a presence or absence of one or more metabolites in the cells.
251. The variant panel of any one of embodiments 235-250, wherein the one or more features of the cells comprise the lipid feature.
252. The variant panel of embodiment 251, wherein the lipid feature is an amount of one or more lipids in the cells, a ratio of at least two lipids in the cells, or a presence or absence of one or more lipids in the cells.
253. The variant panel of any one of embodiments 206-252, wherein the one or more partitions of clonally expanded cells are partitioned on a solid support.
254. The variant panel of any one of embodiments 206-253, wherein the variant panel is produced by the method of any one of embodiments 111-205.
255. A variant panel produced by the method of any one of embodiments 111-205.
256. A system comprising the variant panel of any one of embodiments 206-255.
257. A kit comprising the variant panel of any one of embodiments 206-255.
258. The kit of embodiment 257 further comprising instructions for carrying out methods of any one of embodiments 1 to 205.

### EXAMPLES

### Example 1: Generating of a Variant Panel

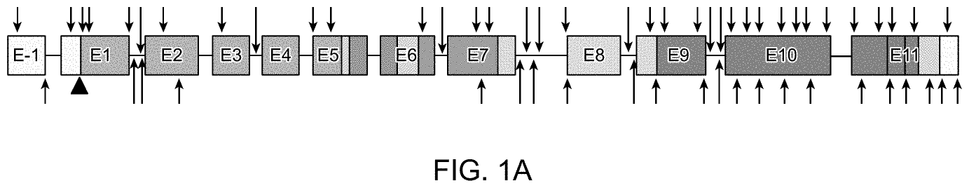

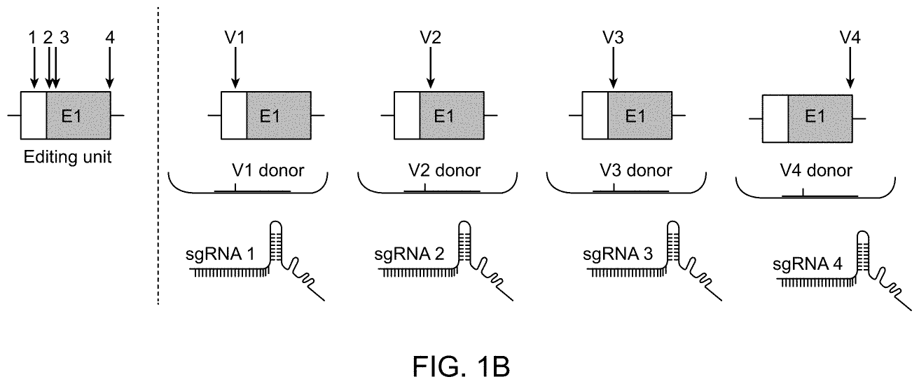

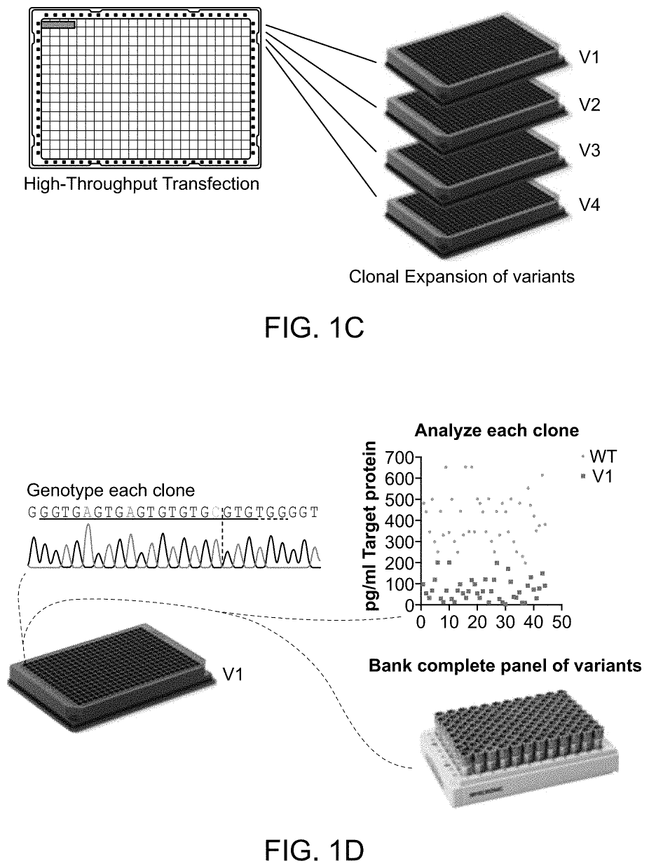

Fifty-eight known variants of a target gene causing a monogenic disease are identified from various online databases as shown in FIG. 1A. For each identified variant, an editing unit made up of a single guide RNA complexed with Cas9 to produce a ribonucleoprotein (RNP) and a donor template containing the desired nucleic acid edit to introduce the variant into the target gene is designed as illustrated in FIG. 1B.

Each of a plurality of pools of cells from an isogenic cell line are contacted with a plurality of editing units designed to introduce a different first nucleic acid edit, thereby producing a plurality of pools of once edited cells. Following the contacting, a subset of the cells from each pool of once edited cells are added into each well of a 384 well plate. FIG. 1C illustrates this process for four variants, V1, V2, V3, and V4). All but one cell in each partition is eliminated via laser photoablation, and the single remaining cell in each partition is allowed to clonally expand.

The genotype of each partition of clonally expanded cells is determined as shown in FIG. 1D. Genotyping of the genomic region of interest in each clonally expanded cell population allows identification of whether the nucleic acid edit is successfully incorporated from the genome, whether additional variants are present, and whether the clonally expanded cell population is the result of a single cell. Clonal populations which successfully incorporated the nucleic acid edit, have no additional variants present, and were clonally expanded from a single cell are added to a variant panel as illustrated in FIG. 1D. The clonal populations added to the variant panel are further analyzed for outcome of the nucleic acid edit. Outcomes of the nucleic acid edit are assessed by determining the amount of protein encoded by the target gene produced by each clone and comparing this amount to the amount of protein encoded by the target gene produced by the original, non-edited isogenic cell line (WT) as shown in FIG. 1D. For the nucleic acid edits which did not successfully incorporate into a clone, the process, from introduction of editing units through genotyping and addition to the variant panel is repeated on new partitions of cells from the same isogenic cell line contacted with newly designed editing units. This process is repeated until the variant panel, contains clonally expanded cells from each of the fifty-eight known variants.

### Example 2: Testing Repair Strategies on a Variant Panel

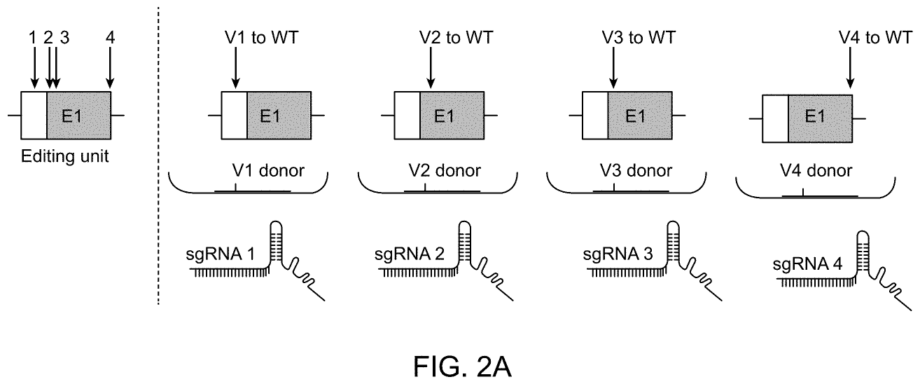

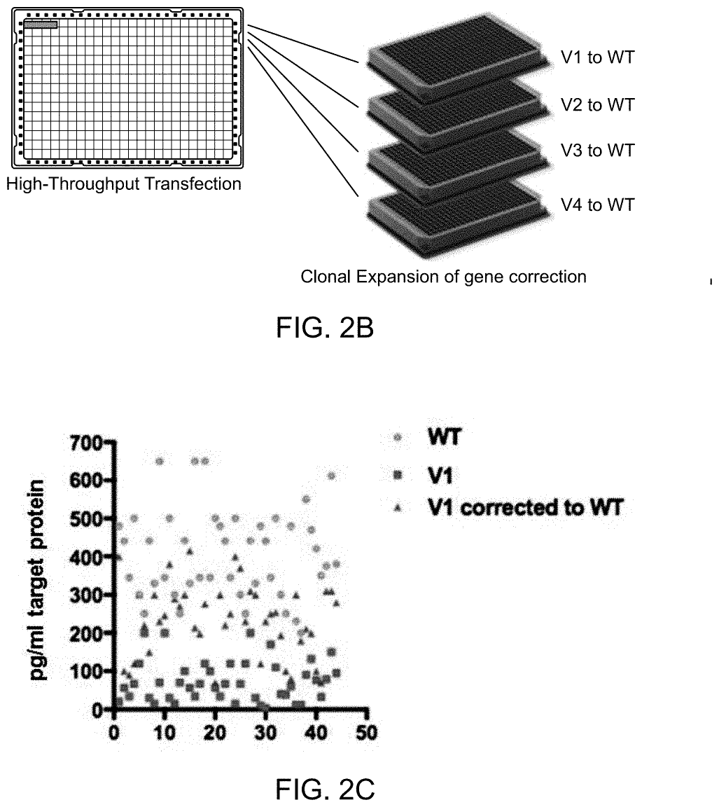

For each variant in the variant panel generated in Example 1, an editing unit made up of a single guide RNA complexed with Cas9 to produce a ribonucleoprotein (RNP) and a donor template containing a nucleic acid edit to repair the variant in the target gene is designed as shown in FIG. 2A.

Into each partition of the variant panel containing the once edited and subsequently clonally expanded cells are added editing units designed to repair the first nucleic acid edit in cells contained in that partition, thereby producing a plurality of twice edited cells All but one cell in each partition containing the plurality of twice edited cells is eliminated via laser photoablation, and the single remaining cell is allowed to clonally expand, thereby producing twice clonally expanded cells as illustrated in FIG. 2B.

The function of each repaired nucleic acid edit is assessed by determining the amount of protein encoded by the target gene produced by each repaired partition of twice clonally expanded cells. The function of the amount of protein encoded by the original, non-edited isogenic cell line (WT) is also determined and compared to the function of each repaired nucleic acid edit as shown in FIG. 2C.

### Example 3: Generation of Variant Panels and Functional Analysis of Clones

FIGS. 3A and 3B illustrate non-limiting examples of embodiments described herein. FIG. 3A illustrates a method for generating a variant panel described herein and FIG. 3B illustrates a method for modifying an outcome of a plurality of first nucleic acid edits described herein.

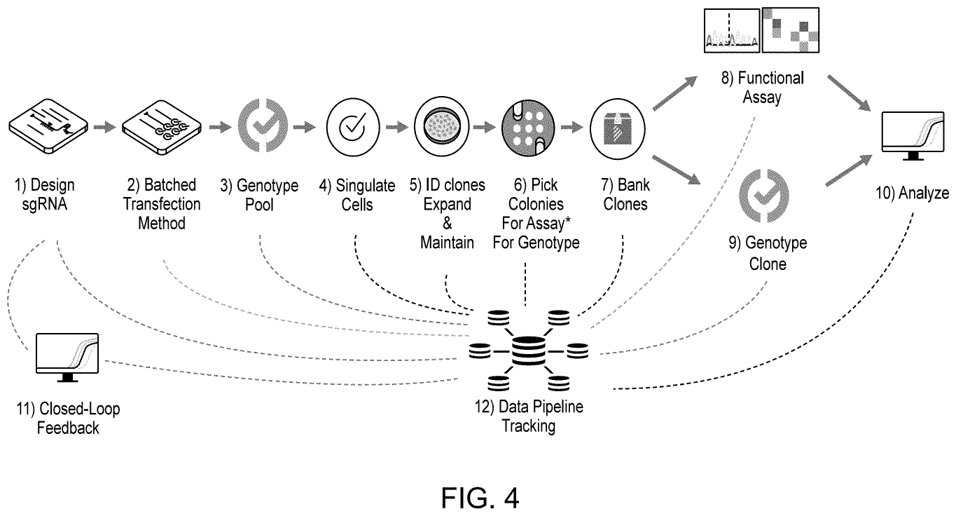

FIG. 4, in steps 1 through 12, describes an embodiment of the methods described herein to generate variant panels and analyze the outcomes of nucleic acid edits. In step 1, sgRNA and DNA single nucleotide variant (SNV) donors are designed by and manufactured internally by Synthego. Step 2 shows simultaneous generation all SNV pools with the sgRNA and DNA donors using Synthego's optimized transfection protocol. Step 3 depicts genotyping analysis of transfected cell pool and determining the SNV knock-in efficiency for each genetic variant. Step 4 shows that single cells from the transfected pool are isolated to generate individual clonal populations for functional analysis. In Step 5, the individual clones are expanded and maintained in the absence of positive or biased phenotype selection. In step 6, individual clones are selected for genotype and phenotype analysis. Step 7 shows that selected clones are subsequently cryobanked and stored, allowing for validation studies or additional functional readout analysis. In step 8, clones undergo functional analysis and, in step 9, genotyping confirmation. Step 10 shows that data analysis is performed to determine the genotype and phenotype correlation. Step 11 shows that the closed-loop feedback bioinformatic analysis allows for continual improvements sgRNA and DNA donor design to generate highly efficient knock-in of the desired edit and subsequently improve the efficiency of the platform to generate SNV clones in a high-throughput manner. In step 12, the data tracking pipeline allows for data collection at the individual clone level for each step. This permits the ability to comprehensively trace a single clone from the guide and donor design, transfection, cloning, expansion and cryobanking all the way through to the phenotypic results.

### Example 4: G6PD Exon 6 Variant Panel Generation

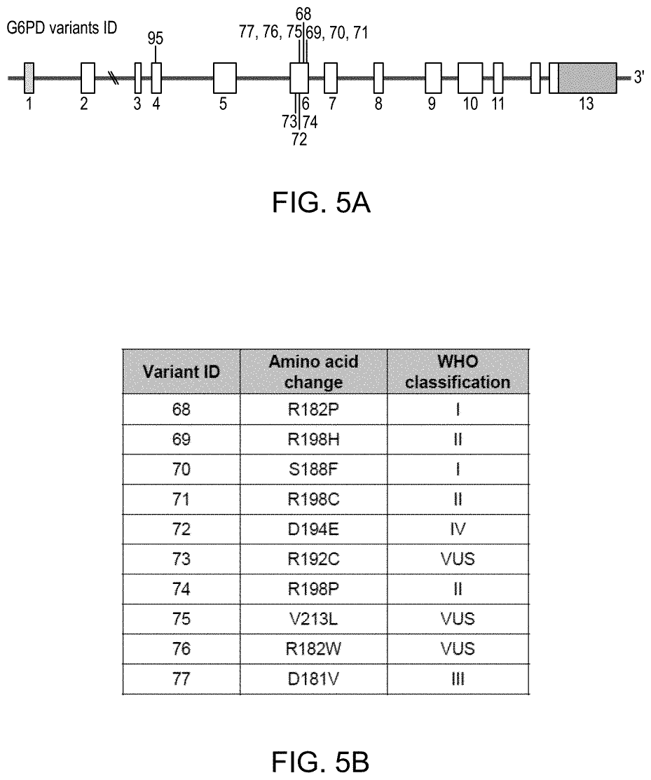

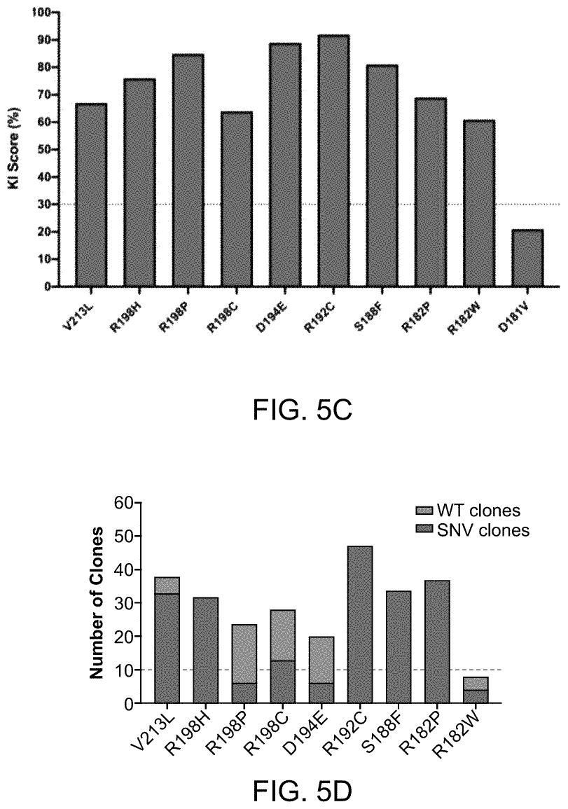

FIGS. 5A-5D describe the G6PD exon 6 variant panel generation. FIG. 5A shows that ten SNVs were identified from the ClinVar database in glucose-6-phosphate-dehydrogenase (G6PD) exon 6. FIG. 5B shows that all G6PD exon 6 SNV are missense mutations. Clinically, these variants range from the most severe (Type I) to normal (Type IV) and three have been identified as variants of unknown clinical significance (VUS). FIG. 5C shows the G6PD clones generated by the Synthego's Engineered Cells platform. Nine out of the 10 variants had a SNV knock in (KI) score >30% and were able to proceed through the single-cell clone generation. FIG. 5D depicts that both homozygous SNV clones and wild type (WT) control clones are generated for functional analysis in the absence of positive phenotype selection. WT control clones refer to those clones that went through the entire clonal workflow but failed to incorporate the SNV knock-in mutations in the G6PD exon 6, and therefore, are genotypically wild type and should exhibit wild type G6PD activity, as observed.

### Example 5: Genotype-Phenotype Analysis of 14 Homozygous Single Nucleotide G6PD Variants

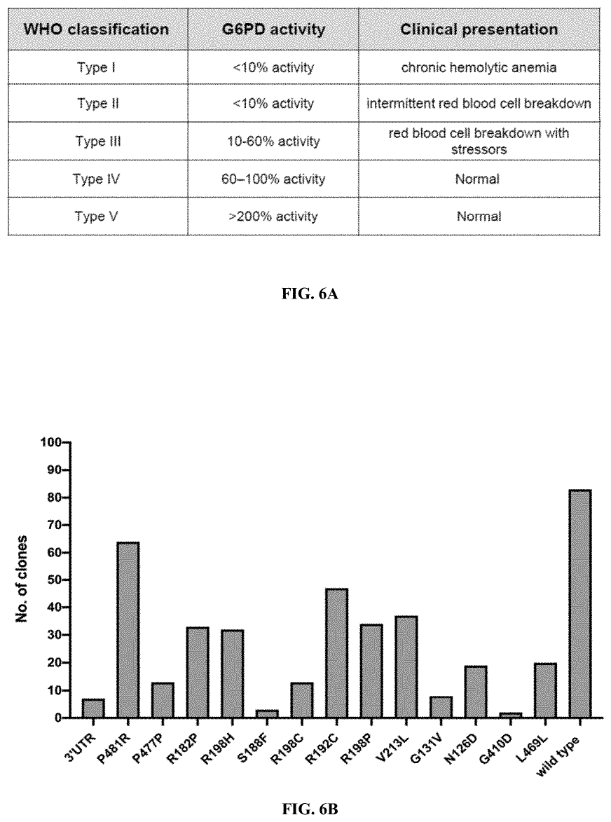

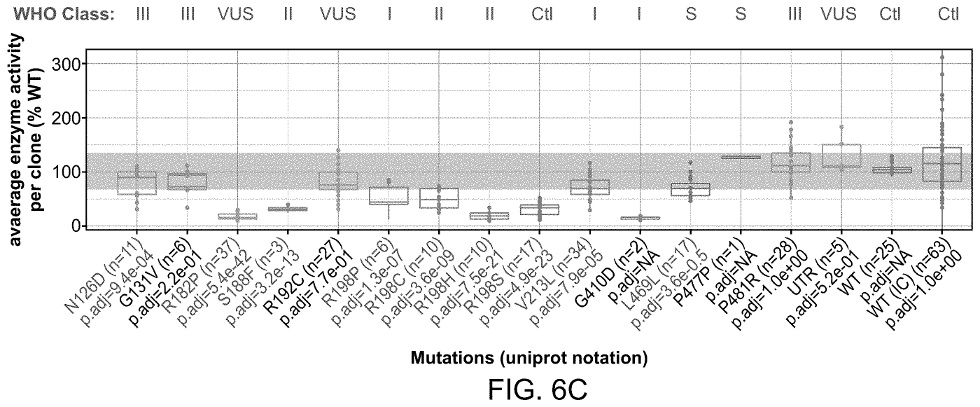

FIG. 6A describes the World Health Organization (WHO) classifies G6PD deficiency into five different types. Type I variants result in the most severe clinical presentation and results from G6PD with less than 10% functional enzymatic activity. Type II variants have less than 10% of wild type G6PD active. Type III variants retain between 10 and 80% functional activity and result in clinical presentation when specified stressors are present. Type IV G6PD have 60 to 100% functional activity with no clinical presentation. Type V have increased enzymatic activity with no clinical consequences. FIG. 6B illustrates Synthego's Engineered Cells platform generated homozygous SNV clones and wild type (WT) control clone for functional analysis. FIG. 6C shows the functional analysis of the 14 G6PD SNV clones generated. Each box plot represents the percent of wild type (WT) activity for an individual clone. The WHO classification is detailed above each variant. In addition, variants of unknown significance (VUS) were also tested in order to identify new clinical classifications for these variants. G6PD R198S Type II variant was used as an internal control. The shaded area is +/−1 standard deviation of wild type clones. Adjusted p-value of each variant is calculated by comparing the distribution of variant clones vs wild type clones. The G6PD variants listed in grey (N126D, R182P, S188F, R198P, R198H, R198S, V213L and L469L) exhibit a significant change in their functional activity as compared to wild type. “Ctl” refers to controls and “S” refers to synonymous mutations.

### Example 6: Identifying Significant Phenotype Variation Between Genetically Identical Clones

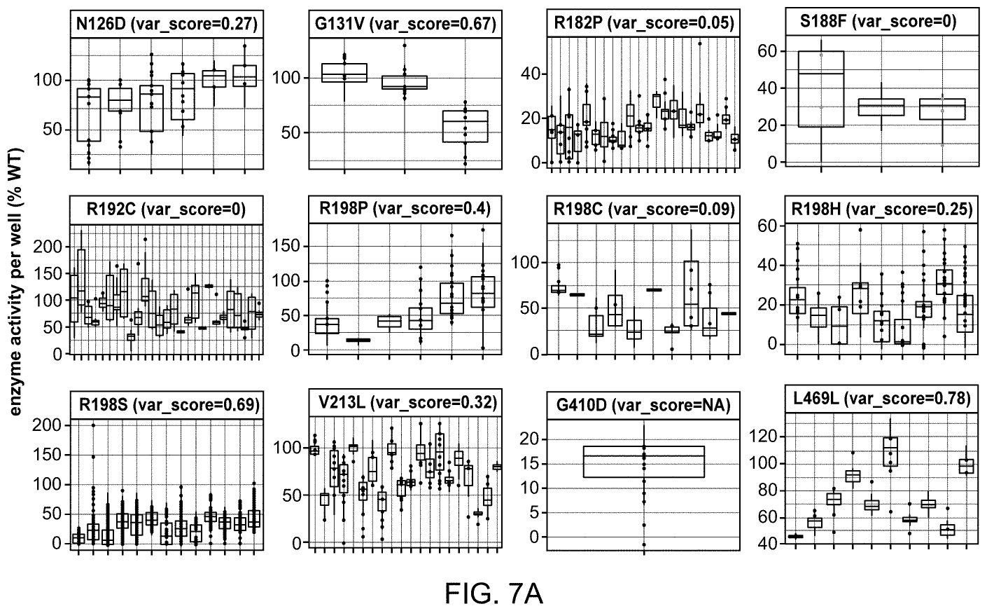

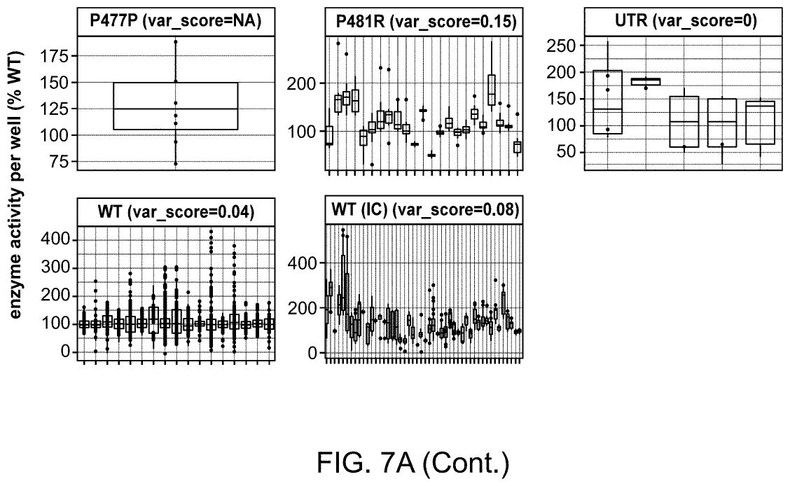

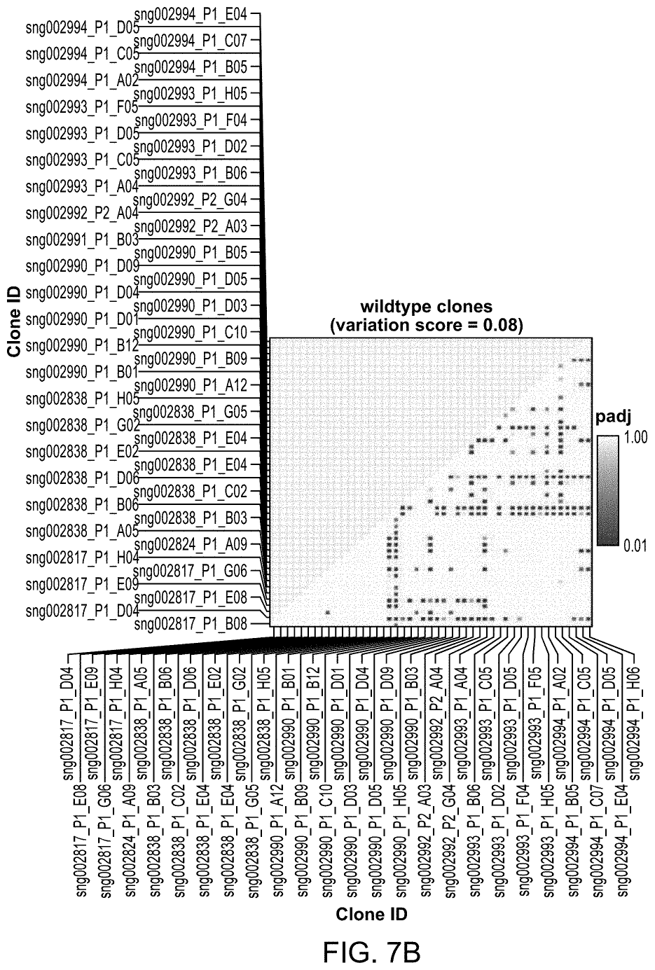

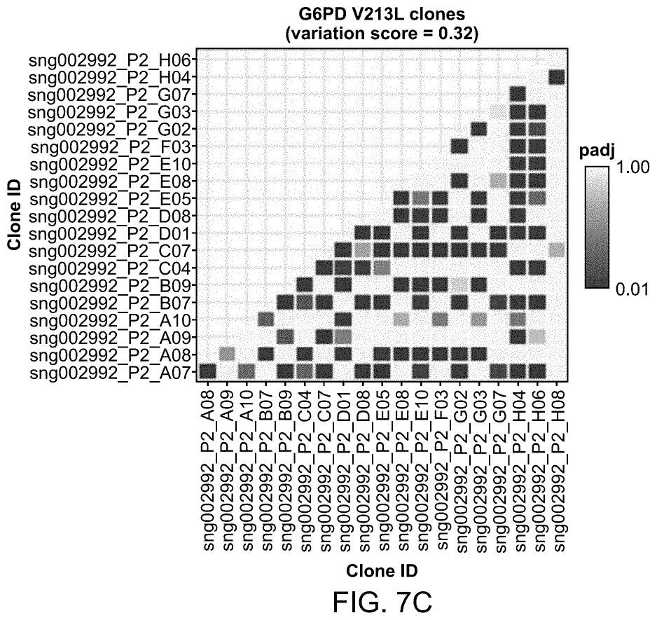

FIGS. 7A-7C illustrate the observed phenotypic variation between genetically identical clones. FIG. 7A graphs represent the enzymatic activity for homozygous clones for the specified G6PD SNV. Each box plot represents the percent of wild type (WT) activity for an individual clone. The variant score (var score) is the measure of differences in G6PD activity between clones. For example, a var score of 0 means that 0% of the pair-wise comparisons have p-values below 0.01, i.e., 0% of the clone-clone comparison are significantly different from each other, a var score of 50 means that 50% of the pair-wise comparisons have p-values below 0.01, i.e., 50% of the clone-clone comparison are significantly different from each other, and a var score of 1 means that 100% of the pair-wise comparisons have p-values below 0.01, i.e., 100% of the clone-clone comparison are significantly different from each other etc. Comparison of all wild type clones and G6PD V213L clones is illustrated in FIG. 7B and FIG. 7C respectively. The graphs represent the adjusted p-value when comparing G6PD functional activity in an individual against all other clones that were identified to have the same genotype at the G6PD locus. The adjusted p-value of each clone is calculated from comparing the distribution for each clone, for example, an adjusted p-value of 0.01 indicates variable functional activity between clones and an adjusted p-value of closer to 1.00 indicates similar functional activity between clones.

### Materials and Methods

### Identification of G6PD SNV

The ClinVar database was used to identify G6PD single nucleotide variants (SNV) within the G6PD locus. The database was accessed on 11 Sep. 2019 and 109 G6PD SNV were identified, of which 72 were classified as a variant of unknown significance.

### Generation of G6PD Knock-in Pools and SNV Clones

The U2OS cells were maintained in McCoy's 5a Medium Modified, supplemented with 10% foetal bovine serum. All 109 SNV knock-in pools were generated by Synthego's Engineered Cells platform using the predetermined optimized transfection protocol for the U2OS cell line. Pools were subjected to genotyping by Sanger sequencing and the knock-in efficiency was determined using Synthego's ICE analysis tool.

Fourteen G6PD variants were selected for clonal functional analysis. Homozygous SNV clones as well as wild type control U2OS clones were generated by Synthego's Engineered Cells platform. The genotype for each clone was determined by Synthego's ICE analysis tool using Sanger sequencing data. Individual clones were maintained in 96 well plates prior to functional assay. Additionally, each 96 well clonal plate was expanded, duplicated and cryopreserved for further analysis.

### Cryopreservation of Clones

Using a EL406 washer dispenser (BioTek), media was removed from the 96 well plates containing the G6PD clones. Wells were rinsed twice with 100 μl phospho-buffered saline (PBS; Gibco)), followed by a rinse with 70 μl of StemPro Accutase Cell Dissociation Reagent (ThermoFisher Scientific). 35 μl of Accutase was added to each well and incubated at 37° C. for 15 minutes. Subsequently, simultaneous quenching and resuspension occurred by addition of 105 μl of complete media supplemented with 13% DMSO. Resuspended cells were transferred to a 96-well round bottom plate, sealed with foil and placed in an insulated polystyrene box at −80° C.

### G6PD Functional Assay

The G6PD assay reagent was developed internally and consists of 50 mM Tris pH7.5 (ThermoFisher), 3.3 mM MgCl2 (Sigma), 100 μM Glucose-6-Phosphate (Sigma), 50 μM Resazurin (Sigma), 10 μM NADP (Sigma), 1 uM YOPRO1(ThermoFisher), 0.1 U/ml Diaphorase (Sigma), and 0.01% v/v Triton X-100 (ThermoFisher).

For measurements of G6PD enzymatic activity, individual clones were seeded to 384 well plates in 30 μl of growth media. After 16h, plates were equilibrated to room temperature for 30 minutes before adding 3 μl of 10x G6PD assay reagent directly to the growth media. Initial fluorescence was measured immediately after addition of the assay reagent (ex540+/−20 nm, em590+/−20 nm, RFUt0) on a BioTek Cytation 5 multimode plate reader using the bottom-read mode. Plates were then incubated at room temperature for 30 minutes, after which a final fluorescence measurement was performed (RFUt1). Assay plates were transferred to a Nexcelom Celigo imager and the number of nuclei present in each well was determined by oxazole yellow iodide staining. To calculate enzymatic activity per cell, data were filtered to exclude wells containing <300 or >2500 cells. Activity per cell was calculated for each well as follows: Activity/cell=(RFUt1−RFUt0)/(elapsed time)*(# of nuclei).

### Data Processing

To avoid unreliable enzyme activity measurements, samples that failed the following two criteria were discarded: First, if the genotype was not an apparent homozygous single nucleotide variant (SNV), determined by either Sanger or NGS reads. Second, if cell counts did not fall between 300 to 2,500 cells per well. For each well, the enzyme activity was first calculated as Relative Fluorescence Unit (RFU) readout divided by total cell count in a given well. This enzyme activity value was further normalized by the wildtype's activity that was taken on the same plate the same date to account for technical variations.

The enzyme activity assay exhibited a decreased signal as the cell count number increases even after the above normalization procedure. Applicant hypothesizes this phenomenon was caused by cell quenching. To address this issue, Applicant assumes that clones of the same mutation type have the same quenching mechanism but with different intensity due to different clones or samples taken on different dates. Briefly, Applicant fit a generalized linear model to samples among the same mutation type with a fixed slope but allow varying intercepts to account for different clones and dates.

For each clone samples taken on the same date, Applicant then calculate a baseline enzyme activity value by plugging in the median cell count in this group to the fitted curve, and subtract each sample's enzyme activities by the delta between their values and the baseline to remove the quenching bias. This procedure was then applied to all mutation types. Finally, this corrected, normalized enzyme activity with the % WT unit is used for the downstream statistical analysis.

### Statistical Analysis

When comparing the phenotypic differences between a given variant to the wildtype clones, Applicant first averaged samples from the same clone. Applicant then fitted a generalized linear model to both the tested variant and wildtype clones with sample dates as an additional variable to adjust for confounding factors. P-value was extracted from the model and adjusted by Benjamini & Hochberg correction (total of 15 variants tested).

To assess variability between clones of a given variant, Applicant performed a pair-wise comparison between two clones by fitting a generalized linear model with sample date as a covariate to adjust for the confounding effect. P-value was extracted from the model and adjusted by Benjamini & Hochberg correction based on the number of pair-wise comparisons in this variant. To summarize a given variant's variability, Applicant further defined a variation score, which is the fraction of pair-wise groups that have adjusted p-value smaller than 0.01.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for determining one or more outcomes of one or more nucleic acid edits, the method comprising:
(a) obtaining one or more partitions of clonal cells, wherein each clonal cell in a partition is clonally expanded from a single cell obtained from contacting one or more original cells with the one or more nucleic acid editing units, and wherein the clonal cells comprise at least one nucleic acid edit in one or more genomic regions of interest; and
(b) determining one or more outcomes of the one or more nucleic acid edits by comparing one or more features of clonal cells in the partition of clonal cells to one or more features of cells in the one or more original cells.

2. The method of claim 1, further comprising comparing one or more features of clonal cells in one partition to one or more features of clonal cells in another partition of a plurality of partitions of clonal cells comprising identical one or more nucleic acid edits in one or more genomic regions of interest.

3. The method of claim 1, wherein the clonal cell is not obtained via a selection.

4. The method of claim 3, wherein the selection is based on one or more of: survival, fitness, expression of a protein and expression of an antibiotic.

5. The method of claim 4, wherein the protein is a fluorescently labeled protein.

6.-47. (canceled)

48. A method for modifying one or more outcomes of one or more first nucleic acid edits in a first genomic region of interest, the method comprising:
(a) obtaining one or more partitions of clonal cells, wherein each partition of clonal cells comprises a first nucleic acid edit from the one or more first nucleic acid edits in a first genomic region of interest, and wherein each clonal cell in a partition is clonally expanded from a single cell obtained from contacting one or more original cells with one or more first nucleic acid editing units; and
(b) contacting each partition of clonal cells with one or more second nucleic acid editing units, wherein each second nucleic acid editing unit is designed to introduce a second nucleic acid edit in a second genomic region of interest thereby producing one or more partitions of twice edited cells, and wherein an outcome of the second nucleic acid edit modifies the outcome of the first nucleic acid edit.

49. The method of claim 48, wherein each twice edited cell in a partition is clonally expanded from a single cell obtained from contacting each partition of clonal cells with one or more second nucleic acid editing units.

50. The method of claim 48, wherein the clonal cell is not obtained via a selection.

51.-110. (canceled)

111. A method for generating a variant panel, the method comprising:
(a) contacting one or more original cells with one or more nucleic acid editing units, wherein each editing unit is designed to introduce at least one nucleic acid edit from one or more nucleic acid edits into one or more genomic regions of interest;
(b) isolating at least one single cell from the one or more original cells contacted with the one or more nucleic acid editing units; and
(c) expanding each single cell in one or more partitions thereby generating one or more partitions of clonal cells.

112. The method of claim 111, wherein the clonal cell is not obtained via a selection.

113. The method of claim 112, wherein the selection is based on one or more of: survival, fitness, expression of a protein and expression of an antibiotic.

114. The method of claim 113, wherein the protein is a fluorescently labeled protein.

115. The method of claim 111, wherein the method comprises contacting one or more original cells in one or more partitions with one or more nucleic acid editing units.

116.-205. (canceled)

206. A variant panel comprising: one or more partitions of clonal cells, wherein each clonal cell in a partition is clonally expanded from a single cell obtained from contacting one or more original cells with the one or more nucleic acid editing units, and wherein the clonal cells comprise at least one nucleic acid edit in one or more genomic regions of interest.

207. The variant panel of claim 206, wherein the clonal cell is not obtained via a selection.

208. The variant panel of claim 207, wherein the selection is based on one or more of: survival, fitness, expression of a protein and expression of an antibiotic.

209. The variant panel of claim 208, wherein the protein is a fluorescently labeled protein.

210. The variant panel of claim 206, wherein the cells in the one or more partitions of clonal cells are isogenic outside of the one or more genomic regions of interest.

211.-258. (canceled)
